SEQUENCE LISTING (4 sequences)

SEQ ID NO: 1 - DNA, 3264 bp, Artificial Sequence
  base sequence for pLSODN-1
agcgcccaatacgcaaaccgcctctccccgcgcgttggccgattcattaatgcagctggcacgacaggtttcccgactggaaagcgggctgtgagcgcaacgcaattaatgtgagttagctcactcattaggcaccccaggctttacactttatgcttccggctcgtatgttgtgtggaattgtgagcggataacaatttcacacaggaaacagctatgaccatgattacgccagcggccgctctagagtcgacggatccgaattcctcgagccatggagatctactagtaagcttcctcagcgaatgcgcagtggcaatgcccgggatgatatcgaagagcctgcaggcatgcggtaccgagctcgggcccgctagcatatgcatgtcttcactggccgtcgttttacaacgtcgtgactgggaaaaccctggcgttacccaacttaatcgccttgcagcacatccccctttcgccagctggcgtaatagcgaagaggcccgcaccgatcgcccttcccaacagttgcgcagcctgaatggcgaatggcgcctgatgcggtattttctccttacgcatctgtgcggtatttcacaccgcatacgtcaaagcaaccatagtacgcgccctgtagcggcgcattaagcgcggcgggtgtggtggttacgcgcagcgtgaccgctacacttgccagcgccttagcgcccgctcctttcgctttcttcccttcctttctcgccacgttcgccggctttccccgtcaagctctaaatcgggggctccctttagggttccgatttagtgctttacggcacctcgaccccaaaaaacttgatttgggtgatggttcacgtagtgggccatcgccctgatagacggtttttcgccctttgacgttggagtccacgttctttaatagtggactcttgttccaaactggaacaacactcaactctatctcgggctattcttttgatttataagggattttgccgatttcggtctattggttaaaaaatgagctgatttaacaaaaatttaacgcgaattttaacaaaatattaacgtttacaattttatggtgcactctcagtacaatctgctctgatgccgcatagttaagccagccccgacacccgccaacacccgctgacgcgccctgacgggcttgtctgctcccggcatccgcttacagacaagctgtgaccgtctccgggagctgcatgtgtcagaggttttcaccgtcatcaccgaaacgcgcgagacgaaagggcctcgtgatacgcctatttttataggttaatgtcatgataataatggtttcttagacgtcaggtggcacttttcggggaaatgtgcgcggaacccctatttgtttatttttctaaatacattcaaatatgtatccgctcatgagacaataaccctgataaatgcttcaataatattgaaaaaggaagagtatgagtattcaacatttccgtgtcgcccttattcccttttttgcggcattttgccttcctgtttttgctcacccagaaacgctggtgaaagtaaaagatgctgaagatcagttgggtgcacgagtgggttacatcgaactggatctcaacagcggtaagatccttgagagttttcgccccgaagaacgttttccaatgatgagcacttttaaagttctgctatgtggcgcggtattatcccgtattgacgccgggcaagagcaactcggtcgccgcatacactattctcagaatgacttggttgagtactcaccagtcacagaaaagcatcttacggatggcatgacagtaagagaattatgtagtgctgccataaccatgagtgataacacagcggccaacttacttctgacaacgatcggaggaccgaaggagctaaccgcttttttgcacaacatgggggatcatgtaactcgccttgatcgttgggaaccggagctgaatgaagccataccaaacgacgagcgtgacaccacgatgcctgtagctatggcaacaacgttgcgcaaactattaactggcgaactacttactctagcttcccggcaacaattaatagactggatggaggcggataaagttgcaggaccacttctgcgctcggcccttccggctggctggtttattgctgataaatctggagccggtgagcgtgggtctcgcggtattattgcagcactggggccagatggtaagccctcccgtatcgtagttatctacacgacggggagtcaggcaactatggatgaacgaaatagacagatcgctgagataggtgcctcactgattaagcattggtaactgtcagaccaagtttactcatatatactttagattgatttaaaacttcatttttaatttaaaaggatctaggtgaagatcctttttgataatctcatgaccaaaatcccttaacgtgagttttcgttccactgagcgtcagaccccgtagaaaagatcaaaggatcttcttgagatcctttttttctgcgcgtaatctgctgcttgcaaacaaaaaaaccaccgctaccagcggtggtttgtttgccggatcaagagctaccaactctttttccgaaggtaactggcttcagcagagcgcagataccaaatactgttcttctagtgtagccgtagttaggccaccacttcaagaactctgtagcaccgcctacatacctcgctctgctaatcctgttaccagtggctgctgccagtggcgataagtcgtgtcttaccgggttggactcaagacgatagttaccggataaggcgcagcggtcgggctgaacggggggttcgtgcacacagcccagcttggagcgaacgacctacaccgaactgagatacctacagcgtgagctatgagaaagcgccacgcttcccgaagggagaaaggcggacaggtatccggtaagcggcagggtcggaacaggagagcgcacgagggagcttccagggggaaacgcctggtatctttatagtcctgtcgggtttcgccacctctgacttgagcgtcgatttttgtgatgctcgtcaggggggcggagcctatggaaaaacgccagcaacgcggcctttttacggttcctggccttttgctggccttttgctcacatgttctttcctgcgttatcccctgattctgtggataaccgtattaccgcctttgagtgagctgataccgctcgccgcagccgaacgaccgagcgcagcgagtcagtgagcgaggaagcggaac

SEQ ID NO: 2 - DNA, 1500 bp, lambda phage
ccaagttagaactcgtcagaatgaatattatcaagcagcaaggcggcatgtttggaccaaataaaaacatctcagaatggtgcatccctcaaaacgagggaaaatcccctaaaacgagggataaaacatccctcaaattgggggattgctatccctcaaaacagggggacacaaaagacactattacaaaagaaaaaagaaaagattattcgtcagagaattctggcgaatcctctgaccagccagaaaacgacctttctgtggtgaaaccggatgctgcaattcagagcggcagcaagtgggggacagcagaagacctgaccgccgcagagtggatgtttgacatggtgaagactatcgcaccatcagccagaaaaccgaattttgctgggtgggctaacgatatccgcctgatgcgtgaacgtgacggacgtaaccaccgcgacatgtgtgtgctgttccgctgggcatgccaggacaacttctggtccggtaacgtgctgagcccggccaaactccgcgataagtggacccaactcgaaatcaaccgtaacaagcaacaggcaggcgtgacagccagcaaaccaaaactcgacctgacaaacacagactggatttacggggtggatctatgaaaaacatcgccgcacagatggttaactttgaccgtgagcagatgcgtcggatcgccaacaacatgccggaacagtacgacgaaaagccgcaggtacagcaggtagcgcagatcatcaacggtgtgttcagccagttactggcaactttcccggcgagcctggctaaccgtgaccagaacgaagtgaacgaaatccgtcgccagtgggttctggcttttcgggaaaacgggatcaccacgatggaacaggttaacgcaggaatgcgcgtagcccgtcggcagaatcgaccatttctgccatcacccgggcagtttgttgcatggtgccgggaagaagcatccgttaccgccggactgccaaacgtcagcgagctggttgatatggtttacgagtattgccggaagcgaggcctgtatccggatgcggagtcttatccgtggaaatcaaacgcgcactactggctggttaccaacctgtatcagaacatgcgggccaatgcgcttactgatgcggaattacgccgtaaggccgcagatgagcttgtccatatgactgcgagaattaaccgtggtgaggcgatccctgaaccagtaaaacaacttcctgtcatgggcggtagacctctaaatcgtgcacaggctctggcgaagatcgcagaaatcaaagctaagttcggactgaaaggagcaagtgtatgacgggcaaagaggcaattattcattacctggggacgcataatagcttctgtgcgccggacgttgccgcgctaacaggcgcaacagtaaccagcataaatcaggccgcggctaaaatggcacgggcaggtcttctggttatcgaaggtaaggtctggcgaacggtgtat

SEQ ID NO: 3 - DNA, 2695 bp, Artificial Sequence
  base sequence for pETUK (del)
gcgcccaatacgcaaaccgcctctccccgcgcgttggccgattcattaatgcaggatctcgatcccgcgaaattaatacgactcactatagggagaccacaacggtttccctctagaaataattttgtttaactttaagaaggagatatacatatgcggggttctcatcatcatcatcatcatggtatggctagcatgactggtggacagcaaatgggtcgggacgatgacgataaggatccccgggtaccgagctcgaattcgatttcgtcgacaagcttagcggccgccgtttaatccggctgctaacaaagcccgaaaggaagctgagttggctgctgccaccgctgagcaataactagcataaccccttggggcctctaaacgggtcttgaggggttttttgctgaaaggaggaactatatccggatctggcgtaatagcgaagaggcccgcaccgatcgcccttcccaacagttgcgcagcctgaatggcgaatggcgcctgatgcggtattttctccttacgcatctgtgcggtatttcacaccgcatctggtgcactctcagtacaatctgctctgatgccgcatagttaagccagccccgacacccgccaacacccgctgacgcgccctgacgggcttgtctgctcccggcatccgcttacagacaagctgtgaccgtctccgggagctgcatgtgtcagaggttttcaccgtcatcaccgaaacgcgcgagacgaaagggcctcgtgatacgcctatttttataggttaatgtcatgataataatggtttcttagacgtcaggtggcacttttcggggaaatgtgcgcggaacccctatttgtttatttttctaaatacattcaaatatgtatccgctcatgagacaataaccctgataaatgcttatgtctgcttacataaacagtaatacaaggggtgttatgagccatattcaacgggaaacgtcttgctcaaggccgcgattaaattccaacatggatgctgatttatatgggtataaatgggctcgcgataatgtcgggcaatcaggtgcgacaatctatcgattgtatgggaagcccgatgcgccagagttgtttctgaaacatggcaaaggtagcgttgccaatgatgttacagatgagatggtcagactaaactggctgacggaatttatgcctcttccgaccatcaagcattttatccgtactcctgatgatgcatggttactcaccactgcgatcccagggaaaacagcattccaggtattagaagaatatcctgattcaggtgaaaatattgttgatgcgctggcagtgttcctgcgccggttgcattcgattcctgtttgtaattgtccttttaacagcgatcgcgtatttcgtctcgctcaggcgcaatcacgaatgaataacggtttggttgatgcgagtgattttgatgacgagcgtaatggctggcctgttgaacaagtctggaaagaaatgcataagctattgccattctcaccggattcagtcgtcactcatggtgatttctcacttgataaccttatttttgacgaggggaaattaataggttgtattgatgttggacgagtcggaatcgcagaccgataccaggatcttgccatcctatggaactgcctcggtgagttttctccttcattacagaaacggctttttcaaaaatatggtattgataatcctgatatgaataaattgcagtttcatttgatgctcgatgagtttttctaattaaaacatatatactttagattgatttaaaacttcatttttaatttaaaaggatctaggtgaagatcctttttgataatctcatgaccaaaatcccttaacgtgagttttcgttccactgagcgtcagaccccgtagaaaagatcaaaggatcttcttgagatcctttttttctgcgcgtaatctgctgcttgcaaacaaaaaaaccaccgctaccagcggtggtttgtttgccggatcaagagctaccaactctttttccgaaggtaactggcttcagcagagcgcagataccaaatactgttcttctagtgtagccgtagttaggccaccacttcaagaactctgtagcaccgcctacatacctcgctctgctaatcctgttaccagtggctgctgccagtggcgataagtcgtgtcttaccgggttggactcaagacgatagttaccggataaggcgcagcggtcgggctgaacggggggttcgtgcacacagcccagcttggagcgaacgacctacaccgaactgagatacctacagcgtgagctatgagaaagcgccacgcttcccgaagggagaaaggcggacaggtatccggtaagcggcagggtcggaacaggagagcgcacgagggagcttccagggggaaacgcctggtatctttatagtcctgtcgggtttcgccacctctgacttgagcgtcgatttttgtgatgctcgtcaggggggcggagcctatggaaaaacgccagcaacgcggcctttttacggttcctggccttttgctggccttttgctcacatgttctttcctgcgttatcccctgattctgtggataaccgtattaccgcctttgagtgagctgataccgctcgccgcagccgaacgaccgagcgcagcgagtcagtgagcgaggaagcggaagt

SEQ ID NO: 4 - DNA, 720 bp, Artificial Sequence
  base sequence for GFP
atggtgagcaagggcgaggagctgttcaccggggtggtgcccatcctggtcgagctggacggcgacgtaaacggccacaagttcagcgtgtccggcgagggcgagggcgatgccacctacggcaagctgaccctgaagttcatctgcaccaccggcaagctgcccgtgccctggcccaccctcgtgaccaccctgacctacggcgtgcagtgcttcagccgctaccccgaccacatgaagcagcacgacttcttcaagtccgccatgcccgaaggctacgtccaggagcgcaccatcttcttcaaggacgacggcaactacaagacccgcgccgaggtgaagttcgagggcgacaccctggtgaaccgcatcgagctgaagggcatcgacttcaaggaggacggcaacatcctggggcacaagttggagtacaactacaacagccacaacgtctatatcatggccgacaagcagaagaacggcatcaaggtgaacttcaagatccgccacaacatcgaggacggcagcgtgcagctcgccgaccactaccagcagaacacccccatcggcgacggccccgtgctgctgcccgacaaccactacctgagcacccagtccgccctgagcaaagaccccaacgagaagcgcgatcacatggtcctgctggagttcgtgaccgccgccgggatcactctcggcatggacgagctgtacaagtaa

(19) United States
(12) Patent Application Publication
(10) Pub. No.: US 2019/0309283 A1
(43) Pub. Date: Oct. 10, 2019

(54) METHOD FOR PREPARING LONG-CHAIN SINGLE-STRANDED DNA

(71) Applicant: BIODYNAMICS LABORATORY, INC., Tokyo (JP)

(72) Inventor: Hitoshi NAGAHORA, Tokyo (JP)

(73) Assignee: BIODYNAMICS LABORATORY, INC., Tokyo (JP)

(21) Appl. No.: 16/318,832

(22) PCT Filed: Jul. 19, 2016
(86) PCT No.: PCT/JP2016/071138

§ 371 Date: Jun. 27, 2019

(51) Int. Cl. C12N 15/10; C12Q 1/6806; C12Q 1/686; C12N 15/64

(52) U.S. Cl. CPC C12N 15/101; C12N 15/64; C12Q 1/686; C12Q 1/6806

(57) ABSTRACT

To provide a method for preparing a long-chain single-stranded DNA that has an accurate sequence having neither internal mutation nor terminal deletion, is homogeneous and is not contaminated with double-stranded DNAs. A target log-chain single-stranded DNA is prepared by: cloning the target DNA using a vector having nicking endonuclease recognition sites or a nicking endonuclease recognition site and a sequence-specific double-strand cleaving endonuclease recognition site; cleaving the vector by using appropriate enzyme(s); electrophoresing the same; and then cutting out a gel that contains the target single-stranded DNA to thereby prepare the target long-chain single-stranded DNA.

### FIELD OF THE INVENTION

The present invention relates to a method for preparing a long-chain single-stranded DNA. Particularly, the present invention relates to a method for using a vector or a target DNA strand having nicking endonuclease recognition site(s) and cleaving the recognition site to thereby prepare a long-chain single-stranded DNA.

### BACKGROUND OF THE INVENTION

Single-stranded DNAs have been used in many molecular biology experiments relating to DNA sequences, SNP analyses, DNA chips, SSCP analyses, SELEX, etc. Several techniques have been used for preparing those single-stranded DNAs, which are largely divided into four techniques. The first technique is chemical synthesis: methods used for chemically synthesizing DNA have recently been improved, so that single-stranded DNAs can be prepared at low cost (Kosuri S, Church G M. (2014) Large-scale de novo DNA synthesis: technologies and applications. Nat Methods. 11(5):499-507). The second technique is a method for using a lambda exonuclease in which phosphoric acid is introduced into a 5′ end of one strand by PCR reaction using a phosphorylated DNA oligomer and then only the phosphorylated strand is decomposed with the lambda exonuclease, so that the other strand of the DNA, which remains undecomposed, can be obtained (Wakimoto Y, Jiang J, Wakimoto H. (2014) Isolation of single-stranded DNA. Curr Protoc Mol Biol. 1; 107:2.15.1-9). The third technique is a method for using biotin in which the biotin is introduced into one stand of a DNA by PCR reaction using a biotinylated DNA oligomer and after alkali denaturation only a target strand is recovered using avidin coated magnetic beads (Avci-Adali M, Paul A, Wilhelm N, Ziemer G, Wendel H P. (2009) Upgrading SELEX technology by using lambda exonuclease digestion for single-stranded DNA generation. Molecules. 24; 15(1):1-11). The fourth technique is a method for using RNA in which a single-stranded DNA is obtained with a reverse transcriptase using RNA as a starting material and then the RNA is decomposed with an RNase to obtain a single-stranded DNA (Miura H, Gurumurthy C B, Sato T, Sato M, Ohtsuka M. (2015) CRISSPR/Cas9-based generation of knockdown mice by intronic insertion of artificial microRNA using longer single-stranded DNA. Sci Rep. 10.1038/srep12799).

However, each of the abovementioned techniques has some problems when actually using it in molecular biology experiments. By way of example, in the first technique (i.e., chemical synthesis), the length of bases that can be synthesized is limited to about 200 bases (Kosuri S, Church G M. (2014) Large-scale de novo DNA synthesis: technologies and applications. Nat Methods. 11(5):499-507); this length is too short to encode the entire gene of general size (about 1,000 bases) (Mashiko D I, Young S A, Muto M, Kato H, Nozawa K, Ogawa M, Noda T, Kim Y J, Satouh Y, Fujihara Y, Ikawa M. (2014) Feasibility for a large scale mouse mutagenesis by injecting CRISPR/Cas plasmid into zygotes. Dev Growth Differ. 56(1):122-9). Moreover, it is known that the error rate of chemical synthesis is very high: while the error rate of replication mechanism in prokaryotes or eukaryotes is 10⁻⁷ to 10⁻⁸, the error rate of typical chemically-synthesized artificial genes is 10⁻² to 10⁻³ (Ma S, Saaem I, Tian J. (2012) Error correction in gene synthesis technology. Trends Biotechnol. 30(3):147-54). When calculated based on these values, 99.998% to 99.9998% of sequence is accurate when a living organism makes a double-stranded DNA having 200 bases, while only 13.4% to 81.9% of sequence is accurate when a double-stranded DNA having 200 bases is made from chemically-synthesized oligomers. Moreover, this error rate of artificially-synthesized genes can only be achieved by employing various error-eliminating techniques available at present, i.e., the error rate might be higher than the abovementioned values when a long-chain single-stranded DNA having 200 bases is simply synthesized chemically (Ma S, Saaem I, Tian J. (2012) Error correction in gene synthesis technology. Trends Biotechnol. 30(3):147-54).

Furthermore, in the second technique using a lambda exonuclease, there are several problems in addition to the problem that synthesized DNA oligomers whose accuracy is not necessarily high must be used, including the problem of introducing mutations into a single-stranded target DNA as a result of performing PCR, the problem of decomposing the single-stranded target DNA by side reactions of the lambda exonuclease, and the problem of leaving lambda exonuclease reaction incomplete (i.e., a double-stranded DNA remains intact) (Wakimoto Y, Jiang J, Wakimoto H. (2014) Isolation of single-stranded DNA. Curr Protoc Mol Biol. 1; 107:2.15.1-9).

In the third technique using biotin, there are also problems similar to the second technique in addition to the problem that synthesized DNA oligomers whose accuracy is not necessarily high must be used, including the problem of possibly introducing mutations into a single-stranded target DNA as a result of performing PCR and the problem of producing incomplete denaturation (i.e., a double-stranded DNA remains intact) at the time of recovering only a target strand using avidin coated magnetic beads after alkali denaturation (Avci-Adali M, Paul A, Wilhelm N, Ziemer G, Wendel H P. (2009) Upgrading SELEX technology by using lambda exonuclease digestion for single-stranded DNA generation. Molecules. 24; 15(1):1-11).

Furthermore, in the fourth technique using RNA, there are also several problems in addition to the problem that synthesized DNA oligomers whose accuracy is not necessarily high must be used, including the problem that the accuracy of reverse transcription reaction using a reverse transcriptase is about the same as the accuracy when a Taq DNA polymerase is used (i.e., the accuracy is not very high, so that mutations could be introduced), the problem that complicated and troublesome steps are required (i.e., acquisition of RNA by transcription, generation of cDNA using a reverse transcriptase and decomposition of RNA using an RNase) and the problem of creating a mixture of normal DNAs and DNAs having deletions because the reverse transcription reaction might be stopped in the middle of those steps (i.e., some 5′ ends are deleted) (Miura H, Gurumurthy C B, Sato T, Sato M, Ohtsuka M. (2015) CRISPR/Cas9-based generation of knockdown mice by intronic insertion of artificial microRNA using longer single-stranded DNA. Sci Rep. 10.1038/srep12799).

### SUMMARY OF THE INVENTION

As genome editing technologies have advanced recently, there is an increasing need for long-chain single-stranded DNAs that cannot be obtained by the abovementioned techniques (Miura H, Gurumurthy C B, Sato T, Sato M, Ohtsuka M. (2015) CRISPR/Cas9-based generation of knockdown mice by intronic insertion of artificial microRNA using longer single-stranded DNA. Sci Rep. 10.1038/srep12799). In genome editing using fertilized eggs of animals such as rats and mice, for example, specific positions or genes on genomes are knocked out using CRISPR-CAS or TALEN or accurate modification and knock-in are performed in small areas such as base substitutions and SNP variants using ssODN (single-strand oligodeoxynucleotide; short-strand single-stranded DNA) as a donor. There is also a desire for modifying larger areas (i.e., knock-in of a specific gene in its entirety or knock-in of the gene of GFP, Cre recombinase or the like in its entirety for the purpose of functional analyses).

In the case of genome editing using fertilized eggs, the efficiency of knock-in must first be improved in order to accomplish the genome editing with high efficiency, because selection cannot be made using a chemical agent, unlike genome editing using cells. However, it is technologically difficult to accurately produce a single-stranded DNA having 200 bases or more by using the conventional ssODN as a donor, though it is very high at the knock-in efficiency and therefore is useful as compared with a method using a double-stranded DNA as a donor (Miura H, Gurumurthy C B, Sato T, Sato M, Ohtsuka M. (2015) CRISPR/Cas9-based generation of knockdown mice by intronic insertion of artificial microRNA using longer single-stranded DNA. Sci Rep. 10.1038/srep12799). Accordingly, there is a need to overcome various quality problems in order to transfer a single-stranded DNA having 200 bases or more to a fertilized egg if it can successfully be produced, because double-stranded DNAs might be mixed, mutations might be introduced terminally or internally or the single-stranded DNA is not homogeneous.

When a target DNA is knocked in by genome editing, the problem is that a double-stranded DNA might be incorporated into sites on the genome other than a target site if the double-stranded DNA is mixed (as pointed out by Mashiko D I, Young S A, Muto M, Kato H, Nozawa K, Ogawa M, Noda T, Kim Y J, Satouh Y, Fujihara Y, Ikawa M. (2014) Feasibility for a large scale mouse mutagenesis by injecting CRISPR/Cas plasmid into zygotes. Dev Growth Differ. 56(1):122-9).

Furthermore, when a target DNA is knocked in by genome editing, the knock-in efficiency is about 10 to 20% at most, so that it takes one to two months to obtain an individual body when a fertilized egg is used. Moreover, since an individual body is used instead of a cell, excessive time and labor must be spent. Accordingly, a great amount of effort is required in order to obtain an accurate knock-in body if mutations and deletions occur at high rates on a single-stranded DNA that should be the basis of genome editing using a fertilized egg.

Accordingly, there has been a need to find a method for preparing a long-chain single-stranded DNA that is homogeneous and is not contaminated with double-stranded DNAs.

The present invention was made in view of the abovementioned circumstances; the object of the present invention is to provide a method for preparing accurate long-chain single-stranded DNAs, which can frequently be used in molecular biology experiments and are also desired in the field of genome editing, by using nicking endonuclease recognition sites when long-chain single-stranded DNAs are prepared.

In order to solve the abovementioned problems, the present inventors examined whether or not it would be possible to prepare a target long-chain single-stranded DNA on the basis of a double-stranded DNA. After conducting intensive studies, they found that a target long-chain single-stranded DNA could be prepared by introducing nicks into a vector, into which a double-stranded DNA having the target single-stranded DNA has been cloned, with a nicking endonuclease and denaturing and then separating the double-stranded DNA.

Specifically, according to a first major point of the present invention, provided is a method for preparing a long-chain single-stranded DNA, the method comprising: (1) a step of cloning a target DNA strand into (a) a vector having at least one nicking endonuclease recognition site on each of both ends of a cloning site, wherein the same strand is cleaved by a nicking endonuclease that recognizes the nicking endonuclease recognition site or into (b) a vector having at least one nicking endonuclease recognition site on one end of a cloning site and at least one sequence-specific double-strand cleaving endonuclease recognition site on the other end of the cloning site; (2) a step of generating at least three types of single-stranded DNAs each having a different molecular weight by cleaving the vector in (a) in which the target DNA strand has been cloned with the nicking endonuclease or by cleaving the vector in (b) in which the target DNA strand has been cloned with the nicking endonuclease and a sequence-specific double-stranded endonuclease that recognizes the sequence-specific double-stranded endonuclease recognition site; (3) a step of denaturing the single-stranded DNA by adding a proper amount of a denaturing agent to the at least three types of single-stranded DNAs; and (4) a step of preparing the target single-stranded DNA by separating the at least three types of denatured single-stranded DNAs with an optional separating means.

Moreover, according to a second major point of the present invention, provided is a method for preparing a long-chain single-stranded DNA, the method comprising: (1) a step of cloning a target DNA strand into a vector, wherein (a) the target DNA has at least one nicking endonuclease recognition site on each of both ends thereof and the same strand is cleaved by a nicking endonuclease that recognizes the nicking endonuclease recognition site or (b) the target DNA has at least one nicking endonuclease recognition site on one end and at least one sequence-specific double-strand cleaving endonuclease recognition site on the other end; (2) a step of generating at least three types of single-stranded DNAs each having a different molecular weight by cleaving the vector in (a) in which the target DNA strand has been cloned with the nicking endonuclease or by cleaving the vector in (b) in which the target DNA strand has been cloned with the nicking endonuclease and a sequence-specific double-stranded endonuclease that recognizes the sequence-specific double-stranded endonuclease recognition site; (3) a step of denaturing the single-stranded DNA by adding a proper amount of a denaturing agent to the at least three types of single-stranded DNAs; and (4) a step of preparing the target single-stranded DNA by separating the at least three types of denatured single-stranded DNAs with an optional separating means.

Such a method allows for providing a single-stranded DNA that has neither mutation nor terminal deletion, has an accurate sequence, is not contaminated with double-stranded DNAs, and has a homogeneous length up to several thousand bases.

Moreover, according to one embodiment of the present invention, the target DNA strand preferably has 200 bases or more in the abovementioned method. Moreover, according to another embodiment of the present invention, the target DNA strand has at least 300 bases, at least 400 bases, at least 500 bases, at least 600 bases, at least 700 bases, at least 800 bases, at least 900 bases or at least 1000 bases.

Moreover, according to another embodiment of the present invention, the optional separating means is gel electrophoresis. In this case, the gel electrophoresis is preferably nondenaturing agarose gel electrophoresis containing no denaturing agent, denaturing agarose gel electrophoresis containing a denaturing agent, nondenaturing acrylamide gel electrophoresis containing no denaturing agent, or denaturing acrylamide gel electrophoresis containing a denaturing agent.

Moreover, according to another embodiment of the present invention, the optional separating means is gel column chromatography. In this case, according to one embodiment of the present invention, the gel column chromatography is preferably gel filtration column chromatography, ion exchange gel column chromatography or affinity gel column chromatography.

Moreover, according to one embodiment of the present invention, the number of bases in the nicking endonuclease recognition site is at least three. Moreover, according to another embodiment of the present invention, the number of bases in the nicking endonuclease recognition site is preferably three, four, five, six or seven. In this case, according to one embodiment of the present invention, the nicking endonuclease is preferably Nb.BbvCI, Nb.BsmI, Nb.BtsI, Nb.BsrDI, Nt.BspQI, Nt.BbvCI, Nt.AIwI, Nt.BsmAI, Nt.BstNBI, Nt.CviPII, Nb.Mva1269I, Nt.Bpu10I or Nb.BssSI.

Moreover, according to another embodiment of the present invention, a guide RNA can bind to the nicking endonuclease recognition site. In this case, the nicking endonuclease is preferably a D10A mutant of Cas9.

Moreover, according to another embodiment of the present invention, the sequence-specific double-stranded cleaving endonuclease recognition site can be a site recognized by an enzyme selected from the group consisting of restriction enzymes and meganucleases or TALEN. In this case, the meganuclease is preferably I-CeuI, I-SceI, PI-PspI or PI-SceI.

Moreover, according to another embodiment of the present invention, a guide RNA or a guide DNA can bind to the sequence-specific double-stranded cleaving endonuclease recognition site. In this case, the sequence-specific double-stranded cleaving endonuclease is preferably Cas9 or Argonaute.

Moreover, according to another embodiment of the present invention, the denaturing agent is formamide, glycerol, urea, thiourea, ethylene glycol or sodium hydroxide. In this case, the denaturing agent is preferably formamide or glycerol.

Moreover, according to a third major point of the present invention, provided is a kit used for the abovementioned first or second major point of the present invention, the kit comprising at least one vector selected from the group consisting of (a) vectors each having at least one nick endonuclease recognition site on each of both ends of a cloning site and (b) vectors each having at least one nicking endonuclease recognition site on one end of a cloning site and at least one sequence-specific double-strand cleaving endonuclease recognition site on the other end of the cloning site.

Moreover, according to one embodiment of the present invention, provided is the abovementioned kit further comprising a reagent containing a denaturing agent used for denaturing DNA.

Moreover, according to a fourth major point of the present invention, provided is a kit used for the abovementioned second major point of the present invention, the kit comprising a vector that does not have a nicking endonuclease recognition site.

Moreover, according to another embodiment of the present invention, provided is the abovementioned kit further comprising a reagent containing a denaturing agent used for denaturing DNA.

The features and marked action and effect of the present invention other than those described above will be clear to those skilled in the art by referring to the following embodiments of the present invention and drawings.

### BRIEF DESCRIPTION OF THE DRAWINGS

FIGS. 1A to 1D are a schematic view of the method according to one embodiment of the present invention.

FIG. 2 is schematic views showing the plasmid map of a vector used in the method according to one embodiment of the present invention (FIG. 2A), the multicloning site in the vector (FIG. 2B), combinations of enzymes at the time of cloning for the purpose of obtaining single-stranded DNAs (FIG. 2C), and the entire sequence (FIG. 2D, SEQ ID NO: 1).

FIG. 3 shows the sequence of a target DNA fragment derived from a lambda phage DNA (1,500 bp, SEQ ID NO: 2), which is used in the method according to one embodiment of the present invention.

FIG. 4 shows the result of electrophoresis obtained by using the method according to one embodiment of the present invention. Formamide was added to a pLSODN-1 (1.5 kb fragment) plasmid with nicks kicked in at two places, and after thermal denaturation, electrophoresis was performed on a 1.2% nondenaturing agarose gel with 1×TAE buffer solution as an electrophoresis carrier solution. On Lane 1, 10 μg of the plasmid was loaded, while 5 μg of the plasmid was loaded on Lane 2.

FIG. 5 shows the result of electrophoresis obtained by using the method according to one embodiment of the present invention. On respective lanes were loaded a pLSODN-1 (1.5 kb fragment) with nicks kicked in at two places (200 ng: Lane 1) and a purified 1.5 kb long-chain single-stranded DNA (200 ng: Lane 2).

FIG. 6 shows the plasmid map of a cloning vector pETUK (del) used in the method according to one embodiment of the present invention (FIG. 6A), the sequence of the multicloning site (FIG. 6B), and the entire sequence (FIG. 6C, SEQ ID NO: 3).

FIG. 7 shows the full length sequence of a GFP gene (720 bases, SEQ ID NO: 4).

FIG. 8 shows the result of electrophoresis obtained by using the method according to one embodiment of the present invention. Formamide was added to a pETUK (GFP-Tyr) plasmid with nicks kicked in at two places, and after thermal denaturation, electrophoresis was performed on a 1.0% 4M urea agarose gel using 4M urea 1×TAE buffer solution as an electrophoresis carrier solution. On Lane 1, DynaMarker DNA High was loaded, while pETUK (GFP-Tyr) without endonuclease treatment and endonuclease-treated pETUK (GFP-Tyr) were loaded on Lane 2 and Lane 3, respectively.

FIG. 9 shows the result of electrophoresing the long-chain single-stranded DNA (759 bases, 100 ng) of a purified GFP gene, which were obtained by using the method according to one embodiment of the present invention.

FIGS. 10A to 10D show the results of electrophoresing plasmids with nicks kicked in at two places on a nondenaturing gel according to one embodiment of the present invention, wherein various types of denaturing agents were used.

### DETAILED DESCRIPTION OF THE INVENTION

The following describes one embodiment and working examples of the present invention with reference to drawings.

As described above, in the method for preparing a long-chain single-stranded DNA according to one embodiment of the present invention, a target long-chain one-stranded DNA is prepared by cloning a target DNA, using a vector having nicking endonuclease recognition sites or a nicking endonuclease recognition site and a sequence-specific double-stranded cleaving endonuclease recognition site, cleaving it with a proper enzyme, electrophoresing the same, and then cutting out a gel that contains the target single-stranded DNA.

In the method for preparing a long-chain single-stranded DNA according to another embodiment of the present invention, a target long-chain one-stranded DNA is prepared by cloning a target DNA having nicking endonuclease recognition sites or a nicking endonuclease recognition site and a sequence-specific double-stranded cleaving endonuclease recognition site into a cloning vector, cleaving it with a proper enzyme, electrophoresing the same, and then cutting out a gel that contains the target single-stranded DNA.

As described above, the problems of the abovementioned four types of conventional methods for preparing single-stranded DNAs are (1) the problem of the difficulty of obtaining a long-chain single-stranded DNA, (2) the problem of sequence inaccuracy and size nonhomogeneity such as internal mutation and terminal deletion, (3) the problem of contamination with double-stranded DNAs, and (4) the problem of complexity associated with operational steps. As described below, it can readily be appreciated that all of the methods according to the present invention are superior to the conventional methods.

First, with regard to the problem of the length of a single-chain DNA in (1), the methods according to the present invention allows for preparing a target long-chain single-stranded DNA by generating three types of DNA molecules each having a different molecular weight using nicking endonuclease(s) or a nicking endonuclease and a sequence-specific double-stranded cleaving endonuclease, and then denaturing and separating the same. In the methods according to the present invention, as described below with regard to the problem (3), the size of a target long-chain single-stranded DNA is preferably about half the size of a vector, when gel electrophoresis is used for separation, in order to prevent a target long-chain single-stranded DNA from being contaminated with other DNA molecules. That is, a target long-chain single-stranded DNA moves toward the tip end of a gel at the time of electrophoresis in such a way that sufficient distances can be achieved from other DNA molecules, so that only a target band can be cut out. Although there is such a restriction on length, a single-stranded DNA having a length of about 6,000 to 7,000 bases can be prepared when a vector having about 12,000 bases is used, for example. The single-stranded DNA having such a length cannot be achieved by the abovementioned conventional methods and is a sufficient length as a sequence for encoding one structural gene. When a cosmid vector (30 kbp to 45 kbp), a BAC vector (about 300 kbp) or a YAC vector (several Mbp) is used, a long-chain single-stranded DNA having a longer length can be prepared using the methods according to the present invention.

Next, with regard to the problem of internal mutation and terminal deletion in (2), since a single-stranded DNA prepared by the methods according to the present invention is derived from a DNA that has been cloned into a vector, the error rate is about the same as that of the replication mechanism of prokaryotes (10⁻⁷ to 10⁻⁸), which is small enough to be disregarded for the normal use of single-stranded DNAs (Ma S, Saaem I, Tian J. (2012) Error correction in gene synthesis technology. Trends Biotechnol. 30(3):147-54). Moreover, a sequence-specific double-stranded cleaving endonuclease such as a restriction enzyme having highly accurate base recognition or a restriction enzyme-derived nicking endonuclease is used for preparing a single-stranded DNA by the methods according to the present invention, and therefore the level of mutation and terminal nonhomogeneity is within the range that can be disregarded for normal experiments.

Moreover, with regard to the problem of being contaminated with double-stranded DNAs in (3), no contamination of double-stranded DNAs occurs at the time of performing electrophoresis by the methods according to the present invention, if the size of a target single-stranded DNA is made about half the size of a vector, the molecular weight of the target single-stranded DNA is prepared in such a way that the target single-stranded DNA can move to the tip end of a gel, and the electrophoresis is performed in such a way that the target single-stranded DNA can sufficiently be separated from other DNAs.

Moreover, with regard to the problem of the complexity of operational steps in (4), the present invention allows for completing the steps of cleaving a vector with nicking endonuclease(s), performing electrophoresis, and cutting out and extracting a band in a half day or within one day at the longest, as long as a DNA is cloned into the vector, because the methods according to the present invention employ a very simple principle.

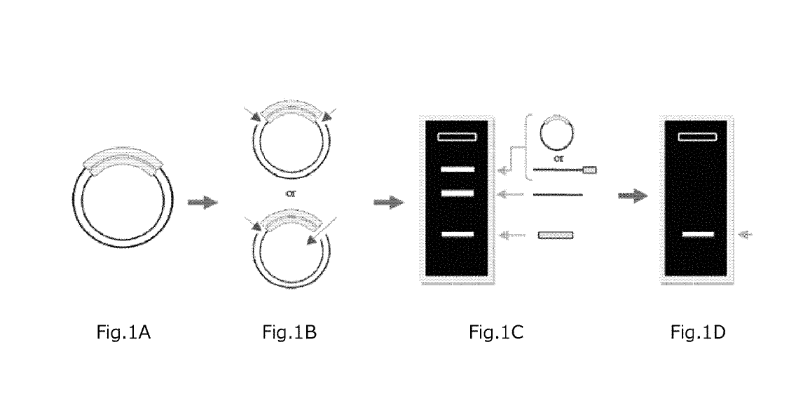

A schematic view showing the flow of the methods according to one embodiment of the present invention is shown in FIG. 1. First, in FIG. 1A, a target DNA to be a long-chain single-stranded DNA is cloned into a vector DNA having nicking endonuclease recognition sites in such a way that the same strand side can be cleaved, wherein the nicking endonuclease recognition sites are positioned at both ends thereof, or a target DNA to be a long-chain single-stranded DNA is cloned into a vector DNA having a nicking endonuclease recognition site and a sequence-specific double-stranded cleaving endonuclease recognition site in such a way that the same strand side can be cleaved, wherein the nicking endonuclease recognition site and the sequence-specific double-stranded cleaving endonuclease recognition site are positioned at respective end of the target DNA. In this case, the vector DNA may have a plurality of nicking endonuclease recognition sites or sequence-specific double-stranded cleaving endonuclease recognition sites at respective end of the cloning site.

Furthermore, in one embodiment of the present invention, a nicking endonuclease recognition site and a sequence-specific double-stranded cleaving endonuclease recognition site may be provided at respective end of a target DNA to be cloned, instead of providing a vector DNA therewith.

Next, in FIG. 1B, a vector into which the abovementioned target DNA has been cloned is cleaved by using nicking endonuclease(s) or a nicking endonuclease and a sequence-specific double-stranded cleaving endonuclease, which can be used for a vector DNA or the target DNA. As a result, three single-stranded DNAs each having a different molecular weight are generated. Two linear sequences and one circular sequence are generated when only a nicking endonuclease is used as a cleaving enzyme, while three linear sequences are generated when nicking endonuclease(s) or a nicking endonuclease and a sequence-specific double-stranded cleaving endonuclease are used.

As a result of cleaving a vector using a nicking endonuclease or a sequence-specific double-stranded cleaving endonuclease, the cleaved sequence is not completely in the single-stranded state but is bound to its complementary strand with hydrogen bonding. Accordingly, after treating a vector using a nicking endonuclease or a sequence-specific double-stranded cleaving endonuclease, a sample obtained in the abovementioned manner is added with a denaturing agent and then subjected to proper treatment for denaturation to make a completely single-stranded DNA. Then, three single-stranded DNAs each having a different molecular weight are subjected to agarose gel electrophoresis to be separated from each other on the basis of molecular weights (FIG. 1C). In this case, the “proper treatment” includes thermal treatment, incubation and overnight still standing; however, any other methods can be used as long as three single-stranded DNAs each having a different molecular weight can be denatured and separated with a denaturing agent. After separating three single-stranded DNAs each having a different molecular weight in this manner, a target single-stranded DNA can be prepared by cutting out and extracting a gel portion containing the target single-stranded DNA (FIG. 1D). As is obvious in FIG. 1B, the target single-stranded DNA is always a sequence having the smallest molecular weight when three single-stranded DNAs each having a different molecular weight is generated by the methods according to the present invention; therefore the lowest band of the gel only have to be cut out after performing electrophoresis.

In the present invention, the term “nicking endonuclease” refers to an endonuclease having nicking activity that can recognize a specific nucleotide sequence and cleave only one strand of a double-stranded nucleic acid having the abovementioned nucleotide sequence. The nicking nuclease can cleave the phosphodiester bond of one strand of a double-stranded DNA. A large number of endonucleases showing such activity have been known together with their recognition sequences, so that those skilled in the art can properly select an endonuclease from among them and use it in the present invention. As used herein, the term “nicking endonuclease” may interchangeably be referred to as “nicking enzyme” or “nickase.”

In one embodiment of the present invention, the number of bases in the nicking endonuclease recognition site is at least 3 or may be 4, 5, 6 or 7. Such endonucleases may include, but not limited to, Nb.BbvCI (the number of bases in the recognition site is 7: 5′-GC/TGAGG-3′), Nb.BsmI (the number of bases in the recognition site is 6: 5′-NG/CATTC-3′), Nb.BtsI (the number of bases in the recognition site is 6: 5′-NN/CACTGC-3′), Nb.BsrDI (the number of bases in the recognition site is 6: 5′-NN/CATTGC-3′), Nt.BspQI (the number of bases in the recognition site is 7: 5′-GCTCTTCN/-3′), Nt.BbvCI (the number of bases in the recognition site is 7: 5′-CC/TCAGC-3′), Nt.AIwI (the number of bases in the recognition site is 5: 5′-GGATCNNNN/N-3′), Nt.BsmAI (the number of bases in the recognition site is 5: 5′-GTCTCN/N-3′), Nt.BstNBI (the number of bases in the recognition site is 5: 5′-GAGTCNNNN/N-3′), Nt.CviPII (the number of bases in the recognition site is 3: 5′-/CCD-3′), Nb.Mva1269I (the number of bases in the recognition site is 6: 5′-G/CATTC-3′), Nt.Bpu10I (the number of bases in the recognition site is 7: 5′-CC/TNAGC-3′) and Nb.BssSI (the number of bases in the recognition site is 6: 5′-C/TCGTG-3′) (the number of bases in the recognition site and its sequence is shown in each parenthesis above). Here, “/” shows a cleavage site; “N” shows A, T, G or C nucleotide; and “D” shows A, T or G nucleotide.

Moreover, as a new artificial nicking endonuclease, CAS9D10A nickase was recently developed by introducing a D10A mutation into CAS9 protein used in genome editing (Ran F A, Hsu P D, Lin C Y, Gootenberg J S, Konermann S, Trevino A E, Scott D A, Inoue A, Matoba S, Zhang Y, Zhang F. (2013) Double nicking by RNA-guided CRISPR Cas9 for enhanced genome editing specificity. Cell. 12; 154(6):1380-9). In one embodiment of the present invention, such an artificially-produced nickase (nicking endonuclease) may also be used.

When the D10A mutation of CAS9 is used as a nicking endonuclease, a target single-stranded DNA can be prepared by making use of the characteristics of the D10A mutation of CAS9, i.e., the introduction of nicks by cleaving only a single strand. Furthermore, when the D10A mutation of CAS9 is used, it is possible to make the “nicking endonuclease recognition site” bind to a guide RNA having a sequence complimentary to the base sequence thereof. A complex of the guide RNA and the D10A mutation of CAS9 recognizes one strand of a double-stranded DNA to introduce nicks. In one embodiment of the present invention, the length of a sequence recognized by a guide RNA is at least five bases, and preferably about 20 bases.

In the present invention, the “sequence-specific double-stranded cleaving endonuclease” could be any endonuclease that can cleave a double strand by recognizing the sequence of a specific DNA and may include a variety of known enzymes or proteins having endonuclease activity. The cleavage mode to be generated could be either a sticking end or a blunt end. For example, in one embodiment of the present invention, the “sequence-specific double-stranded cleaving endonuclease” may include, but not limited to, a variety of restriction enzymes, meganucleases such as I-CeuI, I-SceI, PI-PspI and PI-SceI, and TALEN (a transcription activator-like effector nuclease) that is an artificial nuclease formed by fusing FokI (a restriction enzyme as a DNA cleaving domain) with a TALE protein (as a DNA binding domain) (Cermak T I, Doyle E L, Christian M, Wang L, Zhang Y, Schmidt C, Baller J A, Somia N V, Bogdanove A J, Voytas D F. (2011) Efficient design and assembly of custom TALEN and other TAL effector-based constructs for DNA targeting. Nucleic Acids Res. July; 39(12):e82).

Furthermore, in one embodiment of the present invention, a protein that only has a DNA cleaving capacity and works together with another molecule having a DNA binding capacity can be used. For example, such a protein may include, but not limited to, a CAS9 protein that is dependent on a guide RNA for the DNA binding capacity (Jinek M I, Chylinski K, Fonfara I, Hauer M, Doudna J A, Charpentier E. (2012) A programmable dual-RNA-guided DNA endonuclease in adaptive bacterial immunity. Science. 17; 337(6096):816-21) and an Argonaute protein that is dependent on a guide DNA for the DNA binding capacity (Gao F, Shen X Z, Jiang F, Wu Y, Han C. (2016) DNA-guided genome editing using the Natronobacterium gregoryi Argonaute. Nat Biotechnol. 34(7):768-73). In one embodiment of the present invention, the length of a sequence recognized by a guide RNA or a guide DNA is at least five bases, and preferably about 20 bases.

In the present invention, a vector into which a target DNA strand has been cloned can be cleaved by using nicking endonuclease(s) or a combination of a nicking endonuclease and a sequence-specific double-stranded cleaving endonuclease as described above to generate at least three types of single-stranded DNAs each having a different molecular weight. In this case, sites recognized by the nicking endonuclease or the sequence-specific double-stranded cleaving endonuclease may be either on the vector side or on the target DNA side.

In one embodiment of the present invention, the term “target DNA strand,” “target single-stranded DNA” and “long-chain single-stranded DNA” refers to a DNA having at least 200 bases. Also, in one embodiment of the present invention, the term “target DNA strand,” “target single-stranded DNA” and “long-chain single-stranded DNA” may have at least 300 bases, at least 400 bases, at least 500 bases, at least 600 bases, at least 700 bases, at least 800 bases, at least 900 bases, at least 1,000 bases, at least 1,500 bases, at least 2,000 bases, at least 2,500 bases, at least 3,000 bases, at least 3,500 bases, at least 4,000 bases, at least 4,500 bases, at least 5,000 bases, at least 5,500 bases, at least 6,000 bases, at least 6,500 bases, at least 7,000 bases, at least 7,500 bases, at least 8,000 bases, at least 8,500 bases, at least 9,000 bases, at least 9,500 bases or at least 10,000 bases.

In the present invention, the term “optional separating means” refers to any means capable of separating at least three types of denatured single-stranded DNAs into individual single-stranded DNAs after denaturing at least three types of single-stranded DNAs each having a different molecular weight. By way of example, the optional separating means may include, but not limited to, gel electrophoresis and gel column chromatography.

In one embodiment of the present invention, gel electrophoresis may or may not contain a denaturing agent in a gel or a buffer. By way of example, gel electrophoresis used in one embodiment of the present invention may include, but not limited to, nondenaturing agarose gel electrophoresis containing no denaturing agent, denaturing agarose gel electrophoresis containing a denaturing agent, nondenaturing acrylamide gel electrophoresis containing no denaturing agent, or denaturing acrylamide gel electrophoresis containing a denaturing agent.

In one embodiment of the present invention, when gel electrophoresis is used, a DNA having several thousand bases can be electrophoresed while maintaining it in the single-stranded state simply by adding a physical denaturing agent that lowers Tm to the DNA, subjecting it to thermal denaturing to separate it into the single-stranded DNA state and then loading those single-stranded DNAs onto a nondenaturing gel. By way of example, a double-stranded DNA having 4,751 bases is used in a working example as described below. The present inventors also confirmed that prepared single-stranded DNAs could be electrophoresed while maintaining the single-stranded state even when a double-stranded DNA having 9,547 bases was used. In this case, even when the concentration of plasmid loaded onto an electrophoresis gel is as high as 1 μg/μL a DNA can be denatured to single strands and separated from each other on the basis of difference in molecular weights while maintaining sufficient resolution.

In one embodiment of the present invention, when gel column chromatography is used as the “optional separating means,” any type of gel column chromatography may be used. By way of example, in one embodiment of the present invention, the gel column chromatography may include, but not limited to, gel filtration column chromatography, ion exchange gel column chromatography and affinity gel column chromatography.

Moreover, in one embodiment of the present invention, the term “denaturing agent” refers to a reagent capable of cleaving the hydrogen bond of a double-stranded DNA by means of physical or chemical action to thereby allow separating it to single strands. In the present invention, as described above, a sample obtained by treating a vector using nicking endonuclease(s) or a combination of a nicking endonuclease and a sequence-specific double-stranded cleaving endonuclease cannot always be separated into completely single-stranded DNAs, though nicks are incorporated. Accordingly, a denaturing agent is added to a vector having nicks in order to separate a DNA into three types of single-stranded DNAs each having a different molecular weight. In one embodiment of the present invention, the physical denaturing agent may include, but not limited to, agents that can lower the polarity of a solvent and weaken hydrophobic interaction (e.g., stacking) in the base portion of a nucleic acid such as formamide, glycerol and urea, and sodium hydroxide that is an alkaline reagent capable of inducing the inhibition of hydrogen bond formation by means of deprotonation of bases. Moreover, in one embodiment of the present invention, the chemical denaturing agent may include, but not limited to, agents capable of forming Schiff bases with a nucleic acid such as formaldehyde and glyoxal. Furthermore, in one embodiment of the present invention, thiourea and ethylene glycol may also be used as denaturing agents when electrophoresis is performed.

At the time of electrophoresis, a long-chain RNA is first denatured with a physical denaturing agent such as formamide, the denatured state is fixated covalently with a chemical denaturing agent such as formaldehyde and glyoxal, and then it is electrophoresed in an agarose gel containing formaldehyde. That is to say, RNA needs to be exposed to a chemical denaturing agent not only in a loading buffer but in a gel as well in order to maintain the denatured state. As is assumed on the basis of the electrophoresis of RNA, it should be appreciated that denaturing gel electrophoresis cannot be performed for single-stranded DNAs while preventing reannealing with a complimentary strand and maintaining the single-stranded state, unless an agarose gel added with a chemical denaturing agent is used after fixating denaturation with the same chemical denaturing agent before the electrophoresis. However, there might be some possibility that a covalently-binding denaturing agent causes some types of side reaction though the binding can be removed reversibly. That is to say, the possibility that some mutations might occur in DNAs cannot be denied if a chemical denaturing agent is used. Furthermore, such a chemical denaturing agent is very poisonous and irritable that special precautions are required for handling at the time of actual experiments and the handling is dangerous and inconvenient.

Therefore, in view of safety, convenience and the prevention of DNA damage, it is preferred, in one embodiment of the present invention, that gel electrophoresis be performed in a nondenaturing gel after denaturing a long-chain single-stranded DNA only using a physical denaturing agent.

When electrophoresis is performed with a physical denaturing agent, a single-stranded DNA in a well of an electrophoresis gel leaves the physical denaturing agent contained in a loading buffer and moves into a nondenaturing gel after the electrophoresis was started. The present inventors found that a single-stranded DNA is not reannealed so easily as is conventionally believed after entering the denaturing gel. That is to say, it has been found that a denatured single-stranded DNA can stably be electrophoresed while maintaining the single strand state even in the nondenaturing gel.

The present inventors also found that an single-stranded DNA electrophoresed in a nondenaturing gel was also electrophoresed on the basis of molecular weights and that its resolution was sufficient for the application of the present invention, i.e., the preparation of a single-stranded DNA. A single-stranded DNA in a gel does not have stable structure like double-stranded DNAs or RNAs whose denaturation is fixated with a chemical denaturing agent, and therefore, forms a secondary structure in a gel. Thus, no simple and accurate proportional relationship can be established between the mobility of electrophoresis and the logarithmic values (log₁₀ M) of molecular weights; however, the present invention does not require such a sophisticated mathematical relationship because it is not to analyze subtle differences in molecular weights.

A kit for implementing the methods according to one embodiment of the present invention contains at least one vector selected from the group consisting of (a) vectors each having at least one nicking endonuclease recognition site on each of both ends of a cloning site and (b) vectors each having at least one nicking endonuclease recognition site on one end of a cloning site and at least one sequence-specific double-strand cleaving endonuclease recognition site on the other end of the cloning site. In other words, since both of the abovementioned vectors (a) and (b) have at least one nicking endonuclease recognition site within themselves, a double-stranded DNA to be cloned that is the source of a target single-stranded DNA may or may not have a nicking endonuclease recognition site.

On the other hand, in one embodiment of the present invention, sites recognized by a nicking endonuclease or a sequence-specific double-stranded cleaving endonuclease may consist in both ends of a target DNA to be cloned, as described above; in this case, a vector contained in the kit does not preferably comprise a nicking endonuclease recognition site.

Moreover, in one embodiment of the present invention, the abovementioned kit may contain a denaturing agent used for denaturing DNAs.

### (WORKING EXAMPLES)

The Following Describes the Present Invention in More Detail with Reference to Working Examples; However, the Present Invention is not Limited to Those Working Examples.

Working Example 1 described below shows the cloning procedure of a target DNA when a cloning vector having a nicking endonuclease recognition site positioned at a multicloning site is used; Working Example 2 shows the digestion procedure of the vector into which the target DNA has been cloned with nicking endonucleases (FIG. 1B); and Working Example 3 shows the procedures of subjecting the nicking endonuclease-treated vector into which the target DNA has been cloned to thermal denaturation using a denaturing agent, performing agarose gel electrophoresis under the nondenaturing conditions and then extracting and purifying a target long-chain single-stranded DNA (FIGS. 1C-1D). Working Example 4 shows an example of preparing a long-chain single-stranded DNA containing the entire GFP gene using a vector with nicking endonuclease recognition sites removed from its entire region. Working Example 5 shows the effects of various denaturing reagent on denaturation of pLSODN-1 (1.5Kb fragment) with nicks kicked in at two places.

### (Working Example 1) (Cloning Target DNA into Cloning Vector)

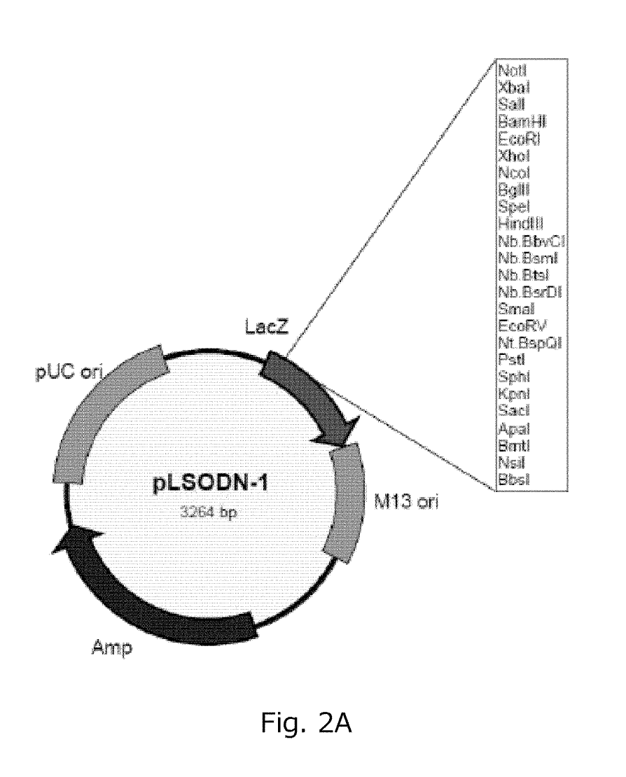

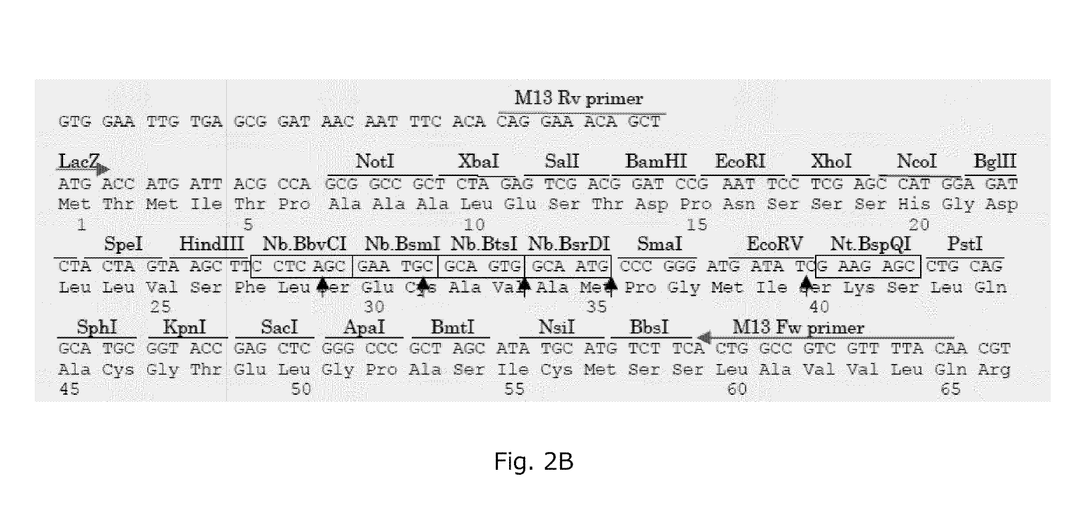

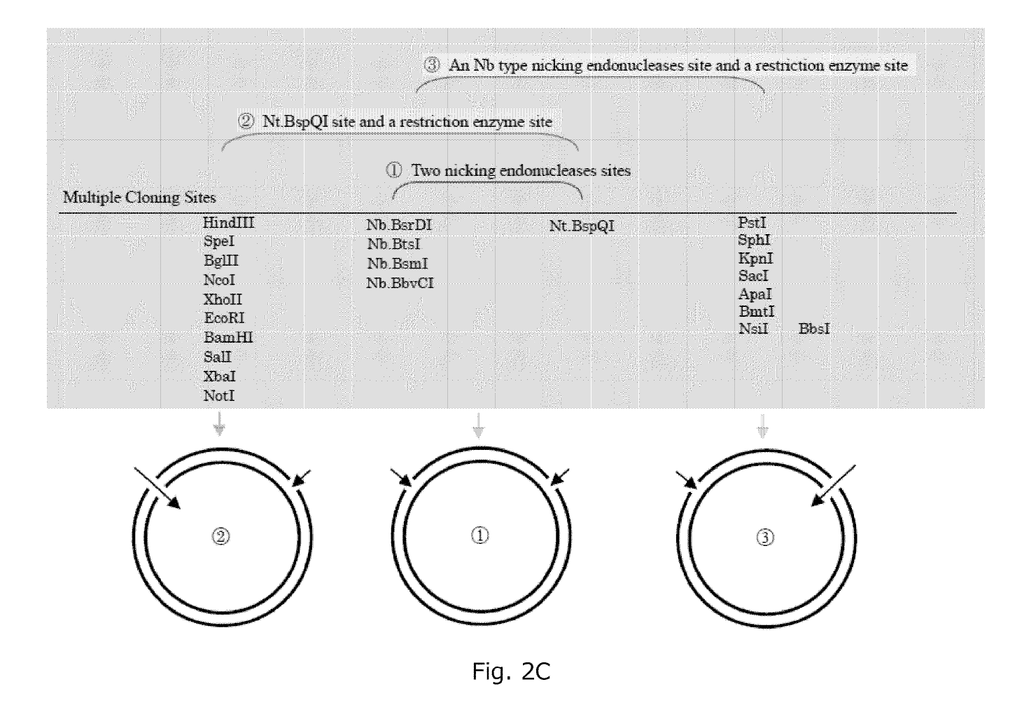

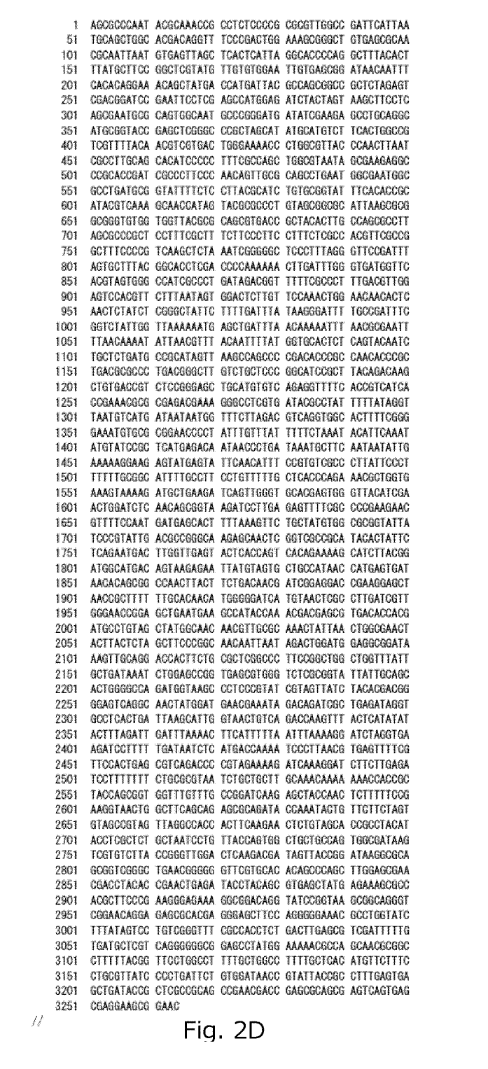

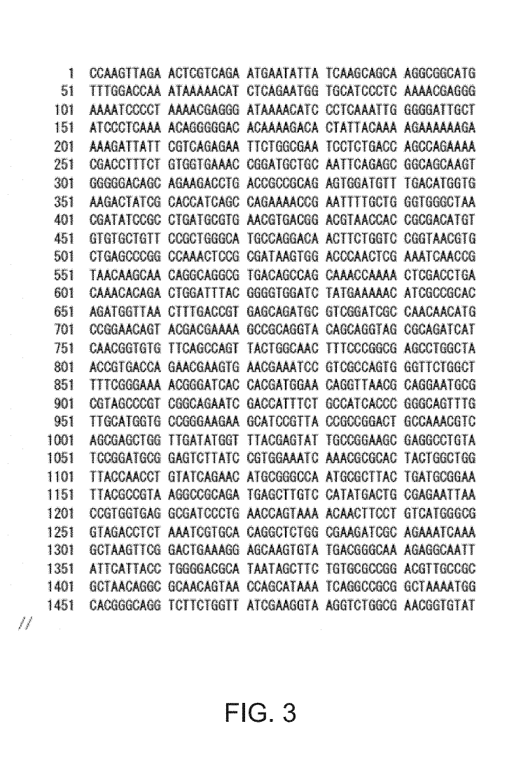

In Working Example 1, a lambda phage-derived 1.5 kb DNA fragment (lambda phage DNA 38,951-40,450, FIG. 3) was used as a model DNA fragment for preparing a long-chain single-stranded DNA. As a cloning vector, pLSODN-1 having a plurality of nicking endonuclease recognition sites positioned at a multicloning site was used. FIG. 2A shows the plasmid map of the cloning vector pLSODN-1; FIG. 2B shows the sequence of the multicloning site; FIG. 2C shows combinations of enzymes at the time of cloning for obtaining single-stranded DNAs; and FIG. 2D shows the base sequence of the entire region. In FIG. 2B, sites shown with boxes are nicking endonuclease sites, wherein nick-introducing positions are shown with arrows. As shown in FIG. 2B, four types of Nb-type nicking endonuclease sites and one type of Nt-type nicking endonuclease site are positioned at the center in such a way that they face each other so that DNA strands on the same side can be cleaved. In the upstream of the Nb-type nicking enzyme sites, a restriction enzyme site recognizing 6 bases or 8 bases for generating a 5′ overhang sticky end was positioned, while in the downstream of the Nt-type nicking enzyme site, a restriction enzyme site recognizing 6 bases for generating a 3′ overhang sticky end was positioned. As shown in FIG. 2C, a target DNA fragment for obtaining a long-chain single-stranded DNA was cloned into a site between a Nb-type nicking enzyme site and a Nt-type nicking enzyme site (circled number 1, a view at the center), a site between a Nt-type nicking enzyme site and a restriction enzyme site recognizing 6 bases for generating a 5′ overhang sticky end (circled number 2, a view on the left) or a site between a Nb-type nicking enzyme and a restriction enzyme site recognizing 6 bases for generating a 3′ overhang sticky end (circled number 3, a view on the right). Here, two nicking endonuclease sites Nb.BsrDI and Nt.BspQI were used.

First, a linear cloning vector pLSODN-1 was obtained by performing PCR for a cloning vector pLSODN-1 using two synthetic DNA oligomers having sequences shown below. The underlined portions in the following sequences are homology sequences of a target DNA that was introduced for the purpose of cloning the target DNA into the vector.

5′ aggtctggcgaacggtgtatcattgccactgcgcattcg 3′
5′ tctgacgagttctaacttggcgaagagcctgcaggcatg 3′

Specifically, PCR was performed in 400 μL in total of a reaction solution prepared by adding 80 pmol of each of the abovementioned two synthetic DNA oligomers, 1×GXL Buffer (Takara Bio Inc.), five units of PrimeSTAR GXL DNA polymerase (Takara Bio Inc.) and 80 nmol of each of dATP, dGTP, dTTP and dCTP to 400 ng of the cloning vector pLSODN-1. The reaction temperature and time are as follows: first, at 95 degrees Celsius for 1 minute; then, 95 degrees Celsius for 1 minutes, 55 degrees Celsius for 1 minute and 72 degrees Celsius for 6 minutes are repeated 16 times in this order; and finally, at 72 degrees Celsius for 10 minutes. This PCR reactant was subjected to 0.8% agarose gel electrophoresis containing 1.6 μg/mL of Crystal Violet. After performing the electrophoresis, a band of 3.2 kb DNA stained in blue was cut out with a razor, purified with the Quiaquick Gel Extraction Kit (QIAGEN, Inc.), dissolved in 50 μL of 10 mM Tris HCl (pH 8.0) and then stored as the linear cloning vector pLSODN-1.

On the other hand, the target DNA used for obtaining a long-chain single-stranded DNA was obtained from a lambda phase DNA by PCR amplification using two synthetic DNA oligomers having sequences shown below. The underlined portions in the following sequences are homology sequences of a vector that was introduced for the purpose of cloning the target DNA into the vector.

5′ cgaatgcgcagtggcaatgatacaccgttcgccagacct 3′
5′ catgcctgcaggctcttcgccaagttagaactcgtcaga 3′

Specifically, PCR was performed in 400 μL in total of a reaction solution prepared by adding 80 pmol of each of the abovementioned two synthetic DNA oligomers, 1×GXL Buffer (Takara Bio Inc.), five units of PrimeSTAR GXL DNA polymerase (Takara Bio Inc.) and 80 nmol of each of dATP, dGTP, dTTP and dCTP to 400 ng of lambda phage DNA (Promega Corporation). The reaction temperature and time are as follows: first, at 95 degrees Celsius for 1 minute; then, 95 degrees Celsius for 1 minutes, 55 degrees Celsius for 1 minute and 72 degrees Celsius for 3 minutes are repeated 16 times in this order; and finally, at 72 degrees Celsius for 10 minutes. This PCR reactant was subjected to 0.8% agarose gel electrophoresis containing 1.6 μg/mL of Crystal Violet. After performing the electrophoresis, a band of 1.5 kb DNA stained in blue was cut out with a razor, purified with the Quiaquick Gel Extraction Kit (QIAGEN, Inc.), dissolved in 50 μL of 10 mM Tris HCl (pH 8.0) and then stored as the target DNA used for obtaining a long-chain single-stranded DNA.

Next, the linear cloning vector pLSODN-1 obtained by PCR amplification was ligated with the lambda phage-derived 1.5 kb target DNA on the basis of the terminal homology sequences introduced by the PCR reaction using the synthetic DNA oligomers.

Specifically, the reaction was carried out at 22 degrees Celsius for 20 minutes in 5 μL in total of a reaction solution prepared by adding 75 ng of the lambda phage-derived 1.5 kb target DNA obtained by PCR amplification, 0.5 μL of 1×Cloning EZ Buffer (GenScript Corporation) and 5 μL of a Clone EZ Enzyme (GenScript Corporation) to 40 ng of the linear cloning vector pLSODN-1 also obtained by PCR amplification, and then the reactant was allowed to stand for 5 minutes on ice to thereby complete the ligation reaction.

Next, the transformation of competent cells (Jet Competent Cell, BioDynamics Laboratory, Inc.) was performed, as shown below, using 1 μL of the ligated reaction solution. First, immediately after melting 25 μL of frozen competent cells on ice, 1 μL of the ligated reaction solution was added to the competent cells, and 5 minutes later the competent cells were transferred to 0.25 mL of Recovery Medium (which comes with Jet Competent Cell) at room temperature. After allowing it to stand for 5 minutes, the bacterial solution suspended in the Recovery Medium was inoculated in an LB agar plate (diameter: 8.5 cm, the amount of the agar medium: 25 mL) containing 50 mg/mL of ampicillin, which was then kept at 37 degrees Celsius for 18 hours. E. coli colonies formed on the LB agar plate as a result of the abovementioned culture were recovered, inoculated to 3 mL of an LB liquid medium containing 50 mg/mL of ampicillin and then subjected to shaking culture at 37 degrees Celsius for 18 hours. Plasmids were prepared from the bacterial cells obtained by the abovementioned shaking culture using Qiagen Plasmid Purification Kit (QIAGEN Inc.).

Plasmids obtained by the abovementioned method were digested with BsrDI and BspQI and then agarose gel electrophoresis analysis was performed to select those having the lambda phase-derived 1.5 kb target DNA fragment accurately inserted into a site between the BsrDI site and the BspQI site of the pLSODN-1 vector by examining their base sequences using ABI PRISM Genetic Analyzer (Applied Biosystems Japan, Ltd.). These selected plasmids were referred to as pLSODN-1 (1.5 kb fragment) plasmids.

### (Working Example 2) (Digestion of Vector Having Target DNA Cloned Using Nicking Enzyme)

The plasmid pLSODN-1 (1.5 kb fragment) prepared in Working Example 1 was digested with nicking endonucleases to cleave both ends only on one side of the lambda phage-derived target 1.5 kb DNA fragment on the abovementioned plasmid having a circular double-stranded DNA structure to thereby introduce nicks.

Specifically, reaction was carried out at 50 degrees Celsius for 60 minutes and then at 60 degrees Celsius for 60 minutes in 50 μL in total of a reaction solution prepared by adding 1×3.1 NEBuffer (New England Biolabs Inc.), 50 units of Nt.BspQI and 50 units of Nb.BsrDI to 100 μL of plasmid pLSODN-1 (1.5 kb fragment). After the reaction, ethanol precipitation was performed as desalination treatment. Specifically, to 50 μL of the pLSODN-1 (1.5 kb fragment) plasmid reaction solution with nicks kicked in at two places was added and mixed with 125 μL of ethanol. Next, this mixed solution was placed in a high-speed microcentrifuge and then rotated at 4 degrees Celsius at 15,000 rpm for 10 minutes for precipitation. After removing the supernatant from the tube, 500 μL of 70% cold ethanol was added to the precipitate; after vortexing, this mixture was placed in the high-speed microcentrifuge and then rotated again at 4 degrees Celsius at 15,000 rpm for 10 minutes for precipitation. The supernatant was removed from the tube by suction; after drying it by placing the tube in a vacuum evaporator, 50 μL of sterile water was added thereto, and after buffer exchange, desalted pLSODN-1 (1.5 kb fragment) plasmids with nicks kicked in at two places was obtained.

### (Working Example 3) (Thermal Denaturation with Denaturing Agent, Separation by Nondenaturing Agarose Gel Electrophoresis, and Extraction and Purification of Gel)

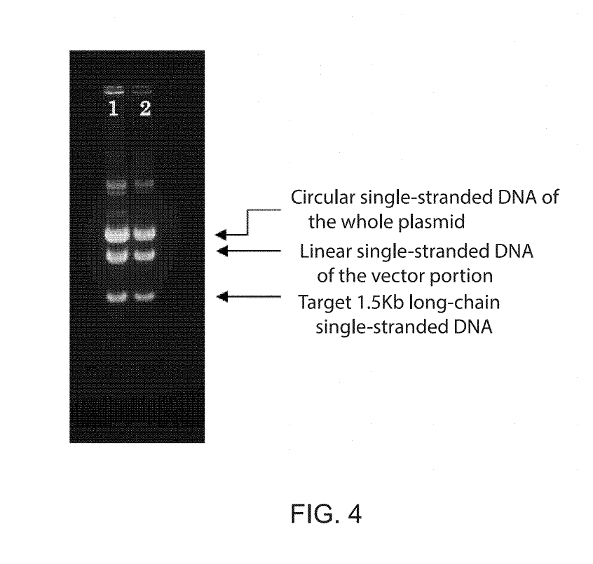

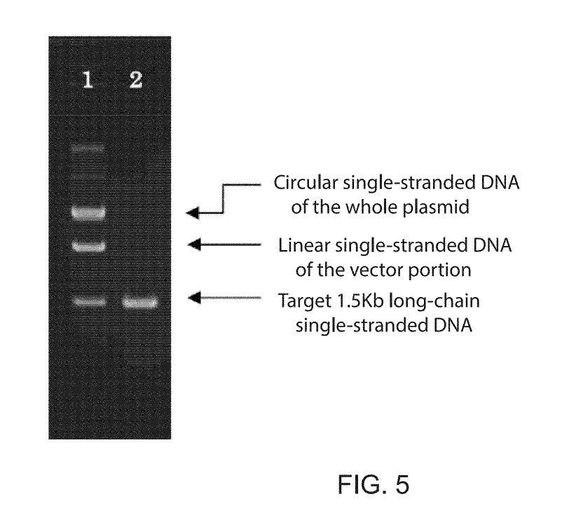

To 10 μL of aqueous solution containing 20 μg of the pLSODN-1 (1.5 kb fragment) plasmids with nicks kicked in at two places was added 95% formamide containing 30 μL of bromophenol blue to prepare a final concentration of 71% formamide solution. Immediately after subjecting this solution to thermal treatment at 70 degrees Celsius for 5 minutes, the solution was placed on ice for rapid cooling. After keeping it on ice for 1 minute, 20 μL (10 μg) and 10 μL (5 μg) were subjected to electrophoresis on a nondenaturing agarose gel having a concentration of 1.2% using 1×TAE Buffer as an electrophoresis carrier solution. Here, please take note that it is not necessary to use a denaturing agarose gel containing urea or formamide. The electrophoresis was stopped when the bromophenol blue pigment moved to proper positions in the gel. After the electrophoresis, the gel was removed from the device, dyed with ethidium bromide and then imaged under ultraviolet light (FIG. 4). After the imaging, out of three bands, a gel piece containing a band of the target 1.5 kb single-stranded DNA that had moved to the tip end and had the smallest molecular weight was cut out. Since the band was at the tip end, there was no possibility that any other bands were mixed. After weighing the gel piece, the target 1.5 kb single-stranded DNA was extracted using QIAquick Gel Extraction Kit (QIAGEN, Inc.). The yield of the target 1.5 kb single-stranded DNA was about 30%. By subjecting a small portion of the purified 1.5 kb single-stranded DNA to the same method, i.e., to the nondenaturing agarose gel electrophoresis on an analystical scale, it was confirmed that the DNA was not contaminated with any other bands and had a high purity (FIG. 5).

### (Working Example 4) (Cloning of GFP Gene into Cloning Vector pETUK (del))

In Working Example 4, a vector pETUK (del) that has no nicking enzyme recognition site in the vector backbone or in the multicloning site was used. Here, nicking endonuclease recognition sites were introduced into both sides of a target DNA fragment by incorporating nicking endonuclease recognition sites into synthetic DNA oligomers, which were used when the vector and the target DNA fragment were amplified by PCR. Moreover, in the present example, a single-stranded DNA encoding the entire GFP gene was prepared. To this GFP gene were bound homology arms constituted of 19 bases on the upstream side of a target place to be inserted into a rat's tyrosinase (Tyr) gene by genome editing and 20 bases on the downstream side of the same. Also, in the present example, an urea agarose gel that is a denaturing gel was used for separating and preparing the single-stranded DNA.

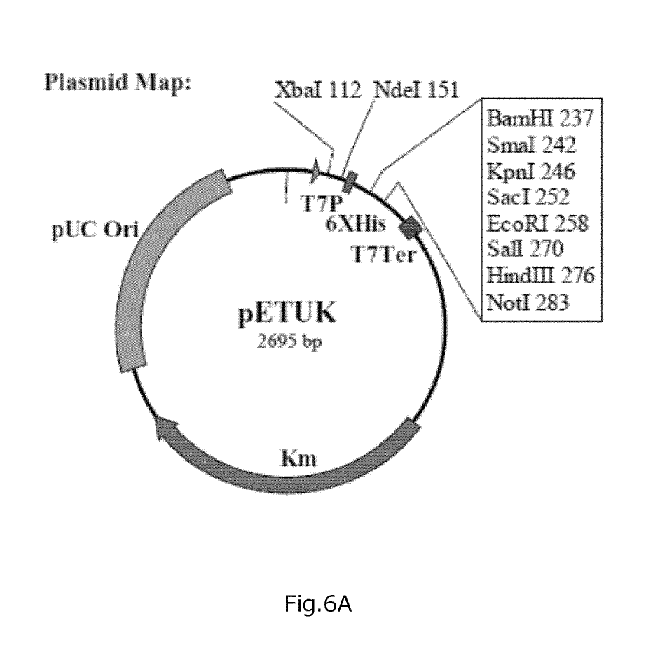

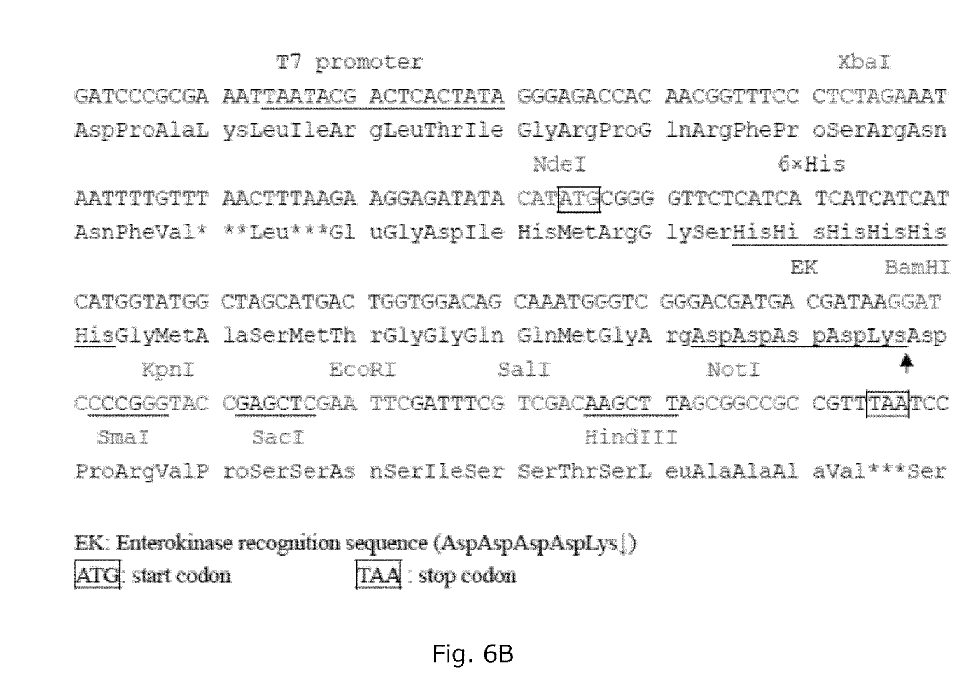

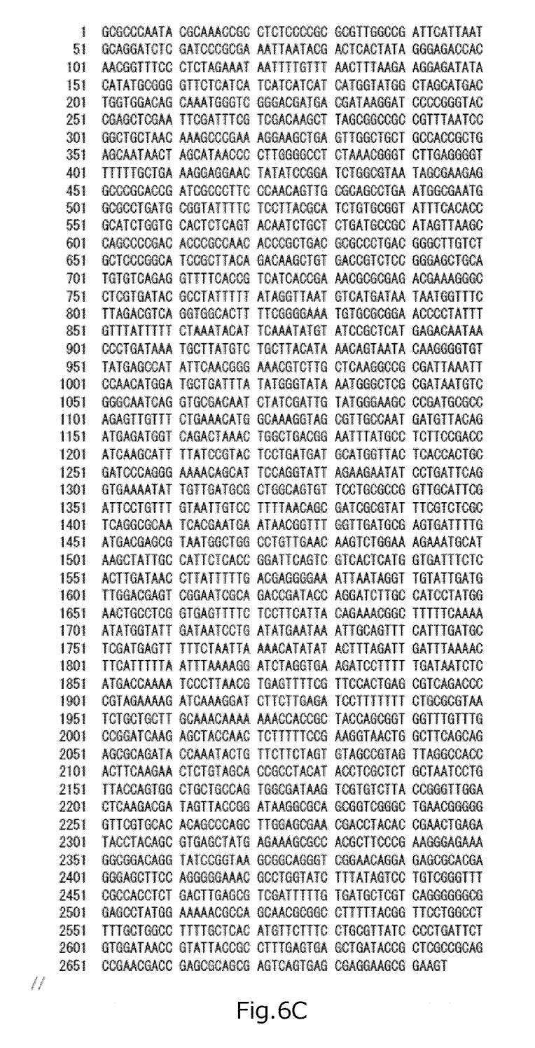

FIG. 6A shows the plasmid map of the cloning vector pETUK (del); FIG. 6B shows the sequence of the multicloning site; and FIG. 6C shows the entire base sequence. Here, two nicking enzyme sites Nb.BsrDI and Nt.BspQI were used for preparing the long-chain single-stranded DNA. As described above, however, the multicloning site of the vector has no such nicking endonuclease site; nicking endonuclease sites were incorporated into synthetic DNA oligomers, which were used when PCR was performed, in order to introduce nicking enzyme recognition sites into both sides of the target DNA.

First, a linear cloning vector pETUK (del) was obtained by performing PCR using two synthetic DNA oligomers having the sequences shown below. The underlined portions in the following sequences are homology sequences of a target DNA that was introduced for the purpose of cloning the target DNA into the vector. The sequences encircled by boxes are those recognized by nicking endonuclease Nb.BsrDI and Nt.BspQI to be introduced into both sides of the target DNA.

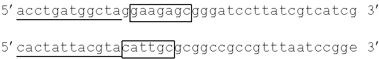

Specifically, PCR was performed in 400 μL in total of a reaction solution prepared by adding 80 pmol of each of the abovementioned two synthetic DNA oligomers, 1×GXL Buffer (Takara Bio Inc.), five units of PrimeSTAR GXL DNA polymerase (Takara Bio Inc.) and 80 nmol of each of dATP, dGTP, dTTP and dCTP to 400 ng of the cloning vector pETUK (del). The reaction temperature and time are as follows: first, at 95 degrees Celsius for 1 minute; then, 95 degrees Celsius for 1 minutes, 55 degrees Celsius for 1 minute and 72 degrees Celsius for 6 minutes are repeated 16 times in this order; and finally, at 72 degrees Celsius for 10 minutes. This PCR reactant was subjected to 0.8% agarose gel electrophoresis containing 1.6 μg/mL of Crystal Violet. After performing the electrophoresis, a band of 2.67 kb DNA stained in blue was cut out with a razor, purified with the Quiaquick Gel Extraction Kit (QIAGEN, Inc.), dissolved in 50 μL of 10 mM Tris HCl (pH 8.0) and then stored as the linear cloning vector pETUK (del).

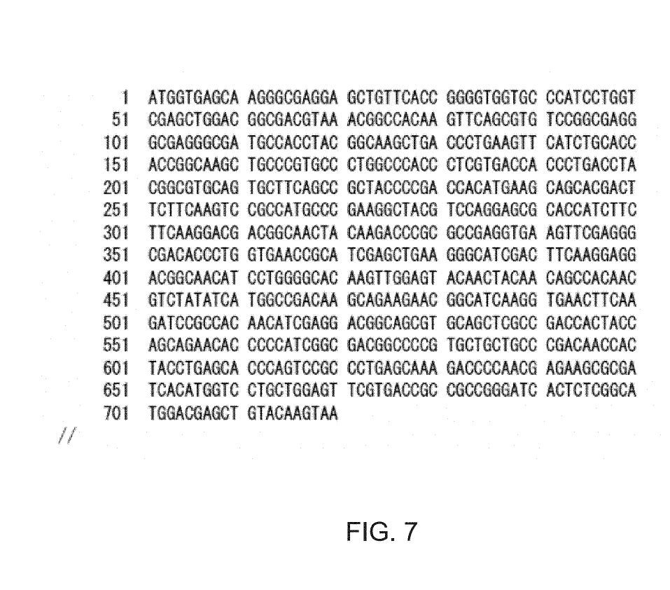

On the other hand, a target GFP gene used for obtaining a long-chain single-stranded DNA was obtained from pCMV-GFP-LC3 Expression vector (Cell Biolabs, Inc.) by performing PCR using two synthetic DNA oligomers having the sequences shown below. The underlined portions in the following sequences are homology sequences of the vector side that was introduced for the purpose of cloning the GFP gene into the vector. The GFP gene fragment is cloned into a site between the BamHI site of the multicloning site of the vector and the NotI site of the same. The sequence of the pCMV-GFP-LC3 Expression vector-derived GFP gene was shown in FIG. 7. The portions encircled by boxes are respectively the start codon and stop codon of GFP protein.

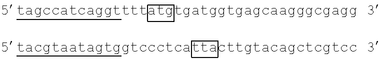

Specifically, PCR was performed in 400 μL in total of a reaction solution prepared by adding 80 pmol of each of the abovementioned two synthetic DNA oligomers, 1×GXL Buffer (Takara Bio Inc.), five units of PrimeSTAR GXL DNA polymerase (Takara Bio Inc.) and 80 nmol of each of dATP, dGTP, dTTP and dCTP to 400 ng of the pCMV-GFP-LC3 Expression vector. The reaction temperature and time are as follows: first, at 95 degrees Celsius for 1 minute; then, 95 degrees Celsius for 1 minutes, 55 degrees Celsius for 1 minute and 72 degrees Celsius for 6 minutes are repeated 16 times in this order; and finally, at 72 degrees Celsius for 10 minutes. This PCR reactant was subjected to 0.8% agarose gel electrophoresis containing 1.6 μg/mL of Crystal Violet. After performing the electrophoresis, a band of 0.75 kb DNA stained in blue was cut out with a razor, purified with the Quiaquick Gel Extraction Kit (QIAGEN, Inc.), dissolved in 50 μL of 10 mM Tris HCl (pH 8.0) and then stored as the DNA fragment of the entire target GFP gene used for obtaining the long-chain single-stranded DNA.

Next, the abovementioned linear cloning vector pETUK (del) fragment obtained by PCR amplification was ligated with the DNA fragment of the entire GFP gene on the basis of the terminal homology sequences introduced by the PCR reaction using the synthetic DNA oligomers.

Specifically, the reaction was carried out at 22 degrees Celsius for 20 minutes in 5 μL in total of a reaction solution prepared by adding 75 ng of the GFP gene obtained by PCR amplification, 0.5 μL of 1× Cloning EZ Buffer (GenScript Corporation) and 5 μL of a Clone EZ Enzyme (GenScript Corporation) to 40 ng of the linear cloning vector pETUK (del) also obtained by PCR amplification, and then the reactant was allowed to stand for 5 minutes on ice to thereby complete the ligation reaction.

Next, the transformation of competent cells (Jet Competent Cell, BioDynamics Laboratory, Inc.) was performed, as shown below, using 1 μL of the ligated reaction liquid. First, immediately after melting 25 μL of frozen competent cells on ice, 1 μL of the ligated reaction liquid was added to the competent cells, and 5 minutes later the competent cells were transferred to 0.25 mL of Recovery Medium (which comes with Jet Competent Cell) at room temperature. After allowing it to stand for 5 minutes, the bacterial solution suspended in the Recovery Medium was inoculated in an LB agar plate (diameter: 8.5 cm, the amount of the agar medium: 25 mL) containing 50 mg/mL of ampicillin, which was then kept at 37 degrees Celsius for 18 hours. E. coli colonies formed on the LB agar plate as a result of the abovementioned culture were recovered, inoculated to 3 mL of an LB liquid medium containing 50 mg/mL of ampicillin and then subjected to shaking culture at 37 degrees Celsius for 18 hours. Plasmids were prepared from the bacterial cells obtained by the abovementioned shaking culture using Qiagen Plasmid Purification Kit (QIAGEN Inc.).

Plasmids obtained by the abovementioned method were digested with BsrDI and BspQI and then agarose gel electrophoresis analysis was performed to select those having the target GFP gene DNA fragment having 759 bases accurately inserted into a site between the BsrDI site and the BspQI site of the pETUK (del) vector by examining their base sequences using ABI PRISM Genetic Analyzer (Applied Biosystems Japan, Ltd.). These selected plasmids were referred to as pETUK (GFP-Tyr) plasmids.

Next, pETUK (GFP-Tyr) plasmids were digested with two nicking endonucleases (NbBsrDI and Nt.BspQI) to introduce nicks into both ends of the GFP gene only on one side thereof. Specifically, reaction was carried out at 50 degrees Celsius for 60 minutes and then at 60 degrees Celsius for 60 minutes in 50 μL of a reaction solution prepared by adding 1×3.1 NEBuffer (New England Biolabs Inc.), 50 units of Nt.BspQI and 50 units of Nb.BsrDI to 100 μg of plasmid pETUK (GFP-Tyr). After the reaction, ethanol precipitation was performed as desalination treatment.

Specifically, to 50 μL of the pETUK (GFP-Tyr) plasmid reaction solution with nicks kicked in at two places was added and mixed with 125 μL of ethanol. Next, this mixed solution was placed in a high-speed microcentrifuge and then rotated at 4 degrees Celsius at 15,000 rpm for 10 minutes for precipitation. After removing the supernatant from the tube, 500 μL of 70% cold ethanol was added to the precipitate; after vortexing, this mixture was placed in the high-speed microcentrifuge and then rotated again at 4 degrees Celsius at 15,000 rpm for 10 minutes for precipitation. The supernatant was removed from the tube by suction; after drying it by placing the tube in a vacuum evaporator, 504, of sterile water was added thereto, and after buffer exchange, desalted pETUK (GFP-Tyr) plasmids with nicks kicked in at two places was obtained.

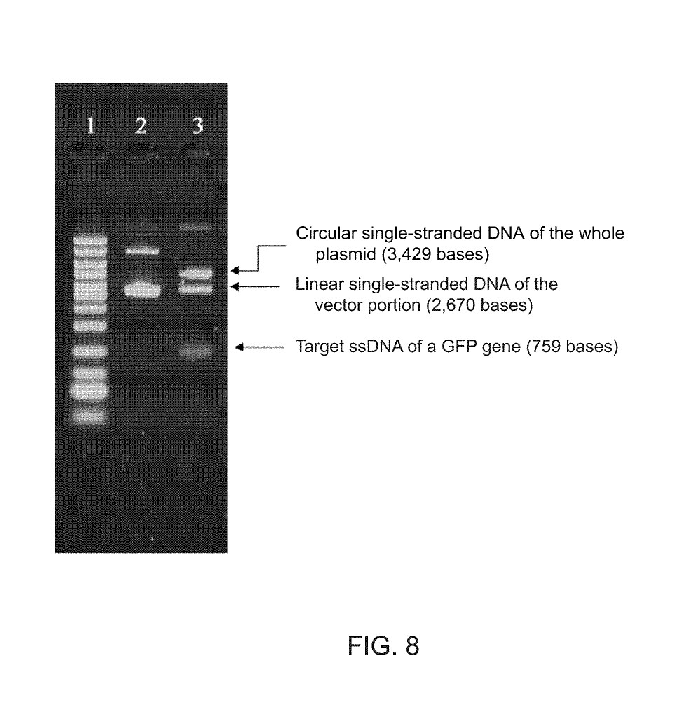

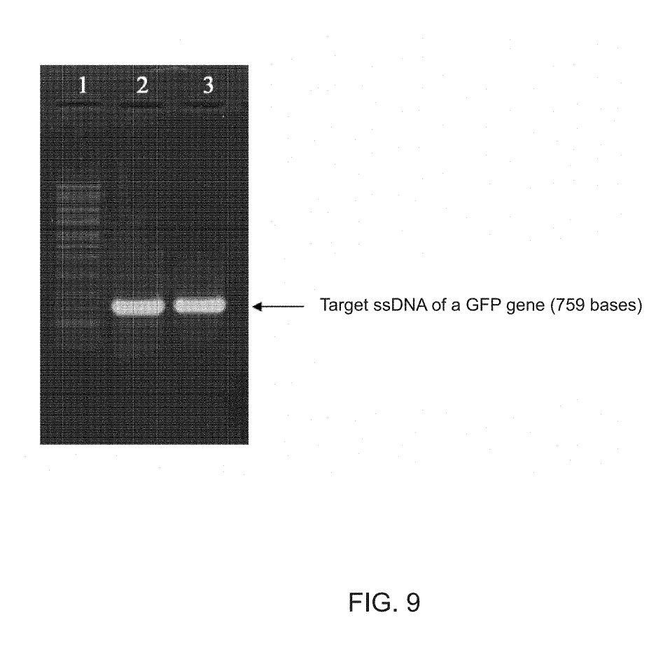

To 10 μL of aqueous solution containing 20 μg of the pETUK (GFP-Tyr) plasmids with nicks kicked in at two places was added 95% formamide containing 304, of bromophenol blue to prepare a final concentration of 71% formamide solution. Immediately after subjecting this solution to thermal treatment at 70 degrees Celsius for 5 minutes, the solution was placed on ice for rapid cooling. After keeping it on ice for 1 minute, a portion thereof (100 ng) was subjected to electrophoresis on a 4M urea agarose gel having a concentration of 1.0% using 1×TAE Buffer containing 4M urea as an electrophoresis carrier solution for the purpose of analysis. The electrophoresis was stopped when the bromophenol blue pigment moved to proper positions in the gel. After the electrophoresis, the gel was removed from the device, dyed with ethidium bromide and then imaged under ultraviolet light (FIG. 8). Since it was confirmed by the analytical electrophoresis that the solution could be separated without a hitch, the remaining amount was subjected to the 4M urea agarose gel electrophoresis. After the electrophoresis, the gel was dyed with ethidium bromide, and out of three bands, a gel piece containing a band of the target GFP single-stranded DNA having 759 bases that had moved to the tip end and had the smallest molecular weight was cut out. Since the band was at the tip end, there was no possibility that any other bands were mixed. After weighing the gel piece, the target target GFP single-stranded DNA having 759 bases was extracted using QIAquick Gel Extraction Kit (QIAGEN, Inc.). The yield of the target GFP single-stranded DNA having 759 bases was about 30%. By subjecting the purified 759 single-stranded DNA to the same method, i.e., to the 4M urea agarose gel electrophoresis, it was confirmed that the DNA was not contaminated with any other bands and had a high purity (FIG. 9).

### (Working Example 5) (Separation of Individual Single-Stranded DNAs by Subjecting pLSODN-1 (1.5 kb Fragment) Plasmids with Nicks Kicked in at Two Places to Denaturing Gel and Nondenaturing Gel Electrophoreses Using Various Denaturing Agents)

The following shows some examples of the appearance and separation of single-stranded DNAs by subjecting pLSODN-1 (1.5 kb fragment) with nicks kicked in at two places to denaturing and nondenaturing gel electrophoreses using some physical denaturing agents. The physical denaturing agents were formamide, glycerol and urea, while sucrose was used as a contrast. Formamide, glycerol and urea are reagents normally used as denaturing agents for electrophoresing nucleic acids.

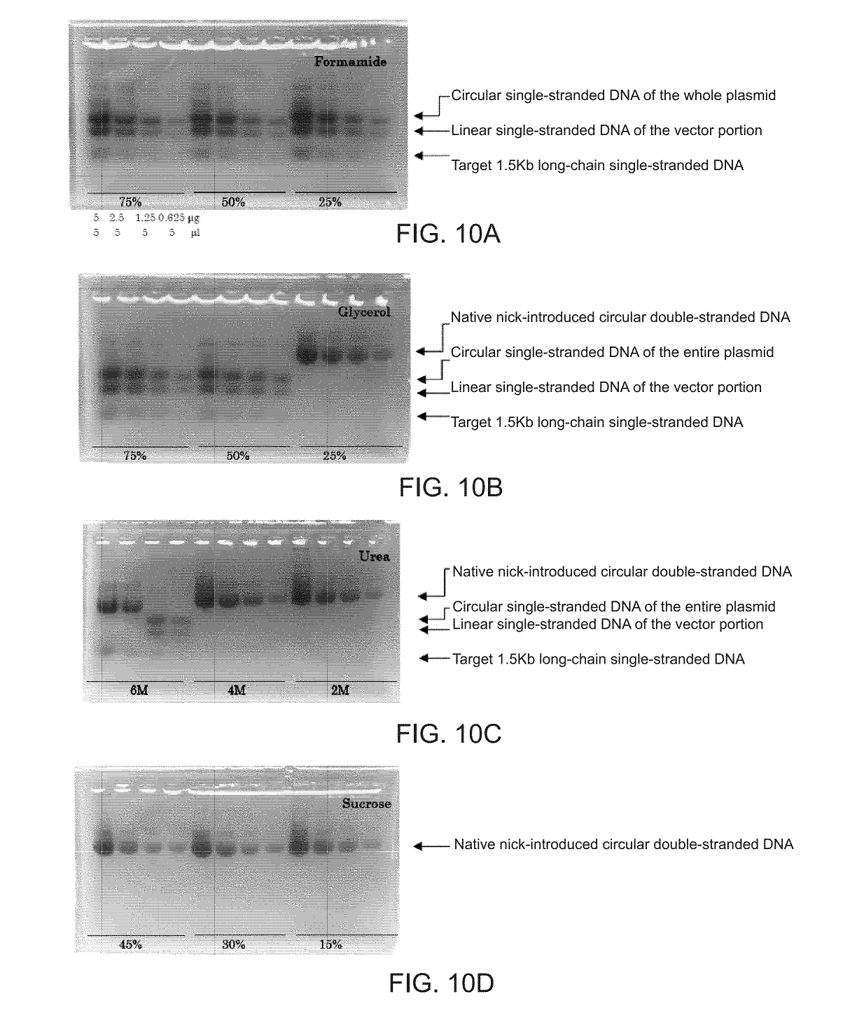

Specifically, various concentrations of pLSODN-1 (1.5 kb fragment) plasmids with nicks kicked in at two places were mixed with various concentrations of formamide, glycerol, urea or sucrose. Next, after heating the mixture at 70 degrees Celsius for 5 minutes, it was quickly cooled on ice. After allowing it to stand on ice for 1 minute, it was subjected to 1.2% agarose gel electrophoresis. After the electrophoresis, the gel was removed and shook together with 1.6% Crystal Violet overnight for staining. FIG. 10 shows the results.

Formamide having a concentration of 25% could completely denature pLSODN-1 (1.5 kb fragment) plasmids with nicks kicked in at two places to give the target 1.5 kb single-stranded DNA, wherein the concentration of plasmids was as high as 1 μg/μL. Glycerol having a concentration of 50% or higher showed the capacity of completely denaturing plasmids. Urea having a concentration of 6M could denature plasmids having a concentration of 0.25 μg/μL; however, the capacity was limited when plasmids had a concentration of 0.5 μg/μL or higher. Sucrose did not show any denaturing effect at all.

It goes without saying that the present invention can be altered in various manners, and therefore a wide variety of modifications are possible without being limited to the abovementioned one embodiment, unless those modifications do not depart from the scope of the present invention.

What is claimed is:

1. A method for preparing a long-chain single-stranded DNA, comprising:
(1) a step of cloning a target DNA strand into (a) a vector having at least one nicking endonuclease recognition site on each of both ends of a cloning site, wherein the same strand is cleaved by a nicking endonuclease that recognizes the nicking endonuclease recognition site or into (b) a vector having at least one nicking endonuclease recognition site on one end of a cloning site and at least one sequence-specific double-strand cleaving endonuclease recognition site on the other end of the cloning site;
(2) a step of generating at least three types of single-stranded DNAs each having a different molecular weight by cleaving the vector in (a) in which the target DNA strand has been cloned with the nicking endonuclease or by cleaving the vector in (b) in which the target DNA strand has been cloned with the nicking endonuclease and a sequence-specific double-stranded endonuclease that recognizes the sequence-specific double-stranded endonuclease recognition site;
(3) a step of denaturing the single-stranded DNA by adding a proper amount of a denaturing agent to the at least three types of single-stranded DNAs; and
(4) a step of preparing the target single-stranded DNA by separating the at least three types of denatured single-stranded DNAs with an optional separating means.

2. A method for preparing a long-chain single-stranded DNA, comprising:
(1) a step of cloning a target DNA strand into a vector, wherein (a) the target DNA strand has at least one nicking endonuclease recognition site on each of both ends thereof and the same strand is cleaved by a nicking endonuclease that recognizes said nicking endonuclease recognition site or (b) the target DNA has at least one nicking endonuclease recognition site on one end and at least one sequence-specific double-strand cleaving endonuclease recognition site on the other end;
(2) a step of generating at least three types of single-stranded DNAs each having a different molecular weight by cleaving the vector in (a) in which the target DNA strand has been cloned with the nicking endonuclease or by cleaving the vector in (b) in which the target DNA strand has been cloned with the nicking endonuclease and a sequence-specific double-stranded endonuclease that recognizes said sequence-specific double-stranded endonuclease recognition site;
(3) a step of denaturing the single-stranded DNA by adding a proper amount of a denaturing agent to the at least three types of single-stranded DNAs; and
(4) a step of preparing the target single-stranded DNA by separating the at least three types of denatured single-stranded DNAs with an optional separating means.

3. The method according to claim 1 or 2, wherein the target DNA strand has 200 bases or more.

4. The method according to claim 1 or 2, wherein the optional separating means is gel electrophoresis.

5. The method according to claim 4, wherein the gel electrophoresis is nondenaturing agarose gel electrophoresis containing no denaturing agent, denaturing agarose gel electrophoresis containing a denaturing agent, nondenaturing acrylamide gel electrophoresis containing no denaturing agent, or denaturing acrylamide gel electrophoresis containing a denaturing agent.

6. The method according to claim 5, wherein the gel electrophoresis is nondenaturing agarose gel electrophoresis containing no denaturing agent.

7. The method according to claim 1 or 2, wherein the optional separating means is gel column chromatography.

8. The method according to claim 7, wherein the gel column chromatography is gel filtration column chromatography, ion exchange gel column chromatography or affinity gel column chromatography.

9. The method according to claim 1 or 2, wherein the number of bases in the nicking endonuclease recognition site is at least three.

10. The method according to claim 9, wherein the nicking endonuclease recognition site is a nicking endonuclease recognition site selected from the group consisting of Nb.BbvCI, Nb.BsmI, Nb.BtsI, Nb.BsrDI, Nt.BspQI, Nt.BbvCI, Nt.AIwI, Nt.BsmAI, Nt.BstNBI, Nt.CviPII, Nb.Mva1269I, Nt.Bpu10I and Nb.BssSI.

11. The method according to claim 10, wherein the number of bases in the nicking endonuclease recognition site is six or seven.

12. The method according to claim 11, wherein the nicking endonuclease recognition site is a nicking endonuclease recognition site selected from the group consisting of Nb.BbvCI, Nb.BsmI, Nb.BtsI, Nb.BsrDI, Nb.BssSI, Nt.BspQI, Nb.Mva1269I, Nt.Bpu10I and Nt.BbvCI.

13. The method according to claim 1 or 2, wherein a guide RNA binds to the nicking endonuclease recognition site.

14. The method according to claim 13, wherein the nicking endonuclease is a D10A mutant of Cas9.

15. The method according to claim 1 or 2, wherein the sequence-specific double-stranded cleaving endonuclease recognition site is a site recognized by an enzyme selected from the group consisting of restriction enzymes and meganucleases or TALEN.

16. The method according to claim 15, wherein the meganuclease is I-CeuI, I-SceI, PI-PspI or PI-SceI.

17. The method according to claim 1 or 2, wherein a guide RNA or a guide DNA binds to the sequence-specific double-stranded cleaving endonuclease recognition site.

18. The method according to claim 17, wherein the sequence-specific double-stranded cleaving endonuclease is Cas9 or Argonaute.

19. The method according to claim 1 or 2, wherein the denaturing agent is formamide, glycerol, urea, thiourea, ethylene glycol or sodium hydroxide.

20. The method according to claim 19, wherein the denaturing agent is formamide or glycerol.

21. A kit used in the method according to claim 1 or 2, the kit comprising at least one vector selected from the group consisting of (a) vectors each having at least one nick endonuclease recognition site on each of both ends of a cloning site and (b) vectors each having at least one nicking endonuclease recognition site on one end of a cloning site and at least one sequence-specific double-strand cleaving endonuclease recognition site on the other end of the cloning site.

22. The kit according to claim 21, further comprising a reagent containing a denaturing agent used for denaturing DNA.

23. The kit used in the method according to claim 2, the kit comprising a vector that does not have a nicking endonuclease recognition site.

24. The kit according to claim 21, further comprising a reagent containing a denaturing agent used for denaturing DNA.
